SEQUENCE LISTING (12 sequences)

SEQ ID NO: 1 - protein, 326 aa, Homo sapiens
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

SEQ ID NO: 2 - protein, 326 aa, Pongo abelii
MPPSISAFQAAYIGIEVLIALVSVPGNVLVIWAVKVNQALRDATFCFIVLLAVADVAVGALVIPLAILINIGPQTYFHTCLMVACPVLILTQSSILALLTIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDNFRCQPAPPIDEDLPEERPDD

SEQ ID NO: 3 - protein, 326 aa, Rattus norvegicus
MPPYISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKTVVTQRRAAVAIAGCWILSLVVGLTPMFGWNNLSVVEQDWRANGSVGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPTCQKPSILIYIAIFLTHGNSAMNPIVYAFRIHKFRVTFLKIWNDHFRCQPKPPIDEDLPEEKAED

SEQ ID NO: 4 - protein, 326 aa, Mus musculus
MPPYISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKTVVTQRRAAVAIAGCWILSLVVGLTPMFGWNNLSEVEQAWIANGSVGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPTCQKPSILIYIAIFLTHGNSAMNPIVYAFRIHKFRVTFLKIWNDHFRCQPKPPIEEDIPEEKADD

SEQ ID NO: 5 - protein, 326 aa, Cavia porcellus
MPHSVSAFQAAYIGIEVLIALVSVPGNVLVIWAVKVNQALRDATFCFIASLAVADVAVGALVIPLAILINIGPQTYFHTCLMVACPVLILTQSSILALLAIAVDRYLRVKIPLRYKTVVTPRRAAVAIAGCWILSLVVGLTPMFGWNNLSKIEMAWAANGSVGEPVIKCEFEKVISMEYMVYFNFFVWVLPPLLLMVLIYLEVFYLIRKQLSKKVSASSGDPQKYYGKELKIAKSLALILFLFALSWLPLHILNCITLFCPTCHKPTILTYIAIFLTHGNSAMNPIVYAFRIQKFRVTFLKIWNDHFRCQPEPPIDEDLPEEKVDD

SEQ ID NO: 6 - protein, 326 aa, Canis lupus
MPPAISAFQAAYIGIEVLIALVSVPGNVLVIWAVKVNQALRDATFCFIVSLAVADVAVGALVIPLAILINIGPRTYFHTCLMVACPVLILTQSSILALLAIAVDRYLRVKIPLRYKTVVTPRRAAVAIAGCWILSFVVGLTPLFGWNRLGEAQRAWAANGSGGEPVIKCEFEKVISMEYMVYFNFFVWVLPPLLLMVLIYLEVFYLIRRQLGKKVSASSGDPQKYYGKELKIAKSLALILFLFALSWLPLHILNCITLFCPSCRKPSILMYIAIFLTHGNSAMNPIVYAFRIQKFRVTFLKIWNDHFRCQPTPPVDEDPPEEAPHD

SEQ ID NO: 7 - protein, 326 aa, Bos taurus
MPPSISAFQAAYIGIEVLIALVSVPGNVLVIWAVKVNQALRDATFCFIVSLAVADVAVGALVIPLAILINIGPRTYFHTCLKVACPVLILTQSSILALLAIAVDRYLRVKIPLRYKTVVTPRRAVVAITGCWILSFVVGLTPMFGWNNLSAVERDWLANGSVGEPVIECQFEKVISMEYMVYFNFFVWVLPPLLLMVLIYMEVFYLIRKQLNKKVSASSGDPQKYYGKELKIAKSLALILFLFALSWLPLHILNCITLFCPSCHMPRILIYIAIFLSHGNSAMNPIVYAFRIQKFRVTFLKIWNDHFRCQPAPPVDEDAPAERPDD

SEQ ID NO: 8 - protein, 12 aa, synthetic construct
GRRRRRRRRRRR

SEQ ID NO: 9 - protein, 10 aa, synthetic construct
GRKKRRQRRR

SEQ ID NO: 10 - protein, 52 aa, synthetic construct
GRKKRRQRRRGSGSGSRIHKFRVTFLKIWNDHFRCQPKPPIEEDIPEEKADD

SEQ ID NO: 11 - protein, 52 aa, synthetic construct
GRKKRRQRRRGSGSGSRIQKFRVTFLKIWNDHFRCQPAPPIDEDLPEERPDD

SEQ ID NO: 12 - protein, 326 aa, unidentified
MPXXXSAFQAAYIGIEVLIALVSVPGNVLVIWAVKVNQALRDATFCFIXXLAVADVAVGALVIPLAILINIGPXTYFHTCLXVACPVLILTQSSILALLXIAVDRYLRVKIPLRYKXVVTXRRAXVAIXGCWILSXVVGLTPXFGWNXLXXXXXXWXANGSXGEPVIXCXFEKVISMEYMVYFNFFVWVLPPLLLMVLIYXEVFYLIRXQLXKKVSASSGDPQKYYGKELKIAKSLALILFLFALSWLPLHILNCITLFCPXCXXPXILXYIAIFLXHGNSAMNPIVYAFRIXKFRVTFLKIWNDXFRCQPXPPXXEDXPXEXXXD

(19) United States
(12) Patent Application Publication
(10) Pub. No.: US 2025/0136658 A1
(43) Pub. Date: May 1, 2025

(54) ANTICONVULSANT AND NEUROPROTECTIVE AGENT

(71) Applicant: THE UAB RESEARCH FOUNDATION, Birmingham, AL (US)

(72) Inventors: Kai JIAO, Augusta, GA (US); Qin WANG, Augusta, GA (US)

(21) Appl. No.: 18/834,738

(22) PCT Filed: Feb. 1, 2023
(86) PCT No.: PCT/US23/61736

§ 371 Date: Jul. 31, 2024

Related U.S. Application Data
(60) Provisional application No. 63/305,621, filed on Feb. 1, 2022.

(51) Int. Cl. C07K 14/705; A61K 38/00; A61K 38/17; A61P 25/08

(52) U.S. Cl. CPC C07K 14/705; A61K 38/1709; A61P 25/08; A61K 38/00; C07K 2319/10

(57) ABSTRACT

Epileptic seizures cannot be effectively controlled in many patients, thereby necessitating the development of novel therapeutic agents. Activation of the A1 receptor (A1R) by endogenous adenosine is an intrinsic mechanism to self-terminate seizures and protect neurons from excitotoxicity. However, targeting A1R for neurological disorders has been hindered by side effects associated with its broad expression outside the nervous system. Herein, the neural-specific A1R/neurabin/RGS4 complex that dictates A1R signaling strength and response outcome in the brain is targeted. A peptide was developed to block the A1R-neurabin interaction and enhance A1R activity. Anticonvulsant and neuroprotective effects are achieved through enhanced A1R function in response to endogenous adenosine in the brain, thus avoiding side effects associated with A1R activation in peripheral tissues and organs.

### CROSS-REFERENCE TO RELATED APPLICATIONS

This application cites the priority of U.S. Patent Application No. 63/305,621, filed on 1 Feb. 2022, which is currently pending. The contents of the foregoing patent application are incorporated by reference in their entirety. The content of the electronic sequence listing (SL_03258-0179.xml; Size 13.6 KB; Date of Creation: Jan. 25, 2023) submitted herewith, is herein incorporated by reference in its entirety.

### STATEMENT REGARDING FEDERAL FUNDING

This invention was made with government support under the National Institutes of Health grant number MH081917. The government has certain rights in the invention.

In this context “government” refers to the government of the United States of America.

### SUMMARY

A novel approach has been developed to produce anticonvulsant effects, neuroprotective effects, or both. The approach involves increasing the activity of the adenosine 1 receptor (A1R) in neural tissue. One aspect of the approach involves reducing the binding of the A1R to neurabin, as neurabin reduces the activity of A1R. An agent has been developed that in some embodiments is useful to produce anticonvulsant effects, neuroprotective effects, or both, according to this approach, comprising a C-terminal region of the A1R. It appears that the agent interrupts binding between A1R and neurabin without otherwise disturbing the functions of these proteins.

### BRIEF DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure.

FIG. 1A shows a graphical representation of neurabin deficiency (Ppp1r9a^{-/-}) attenuating seizure severity in response to PTZ. Seizure activity over time following PTZ injection was scored. N=8-9/group. For Ppp1r9a^{-/-} vs. WT mice, **** represents p<0.0001 by two-way ANOVA. For WT mice treated with PTZ alone vs. PTZ plus DPCPX, **** represents p<0.0001 by two-way ANOVA.

FIG. 1B shows a graphical representation of PTZ-induced lethality within 40 min post injection for the mice examined in FIG. 1A. The number of mice in each group is indicated in parentheses.

FIG. 1C shows a graphical representation of seizure severity in response to kainate measured in the Rgs4^{-/-} mouse line and its corresponding WT line. N=8-14/group. For Rgs4^{-/-} vs. WT mice ** represents p<0.01. For WT mice treated with kainate alone vs. kainate plus DPCPX, #### represents p<0.0001 by two-way ANOVA.

FIG. 1D shows a graphical representation of kainate-induced lethality in mice examined in FIG. 1C. Data are expressed as percentage of death in WT and Rgs4^{-/-} mice caused by administration of kainate alone or with DPCPX. The number of mice in each group is indicated in parentheses.

FIG. 1E shows a graphical representation of A1R-mediated anticonvulsant effects that are enhanced in mice with reduced neurabin expression. Seizure severity in response to kainate is attenuated in both Ppp1r9a^{+/-} and Ppp1r9a^{-/-} mice. The number of mice in each group is indicated in parentheses. For Ppp1r9a^{+/-} vs. Ppp1r9a^{+/+} mice, **** represents p<0.0001 by two-way ANOVA.

FIG. 1F shows a representative Western blot and graphical representation of the expression of neurabin in brain lysates of Ppp1r9a^{+/+}, Ppp1r9a^{+/-} and Ppp1r9a^{-/-} mice. Top, representative Western blots. Bottom, quantitation of expression levels of neurabin in the brain of mice with indicated genotypes. For Ppp1r9a^{-/-} vs. Ppp1r9a^{+/+} mice, **** represents p<0.0001 by one-way ANOVA. N=4/group. Data are presented as mean±SEM.

FIG. 2A shows a graphical representation of traces of field excitatory postsynaptic potentials (fEPSP) at CA3-CA1 synapses in hippocampal slices from WT mice treated with different concentrations of R-PIA.

FIG. 2B shows a representative summary plot of mouse hippocampal slices exposed to R-PIA (min 20-50) showing the reduction of the fEPSP during bath application of 10 nM (n=5), 30 nM (n=4) and 100 nM (n=4) R-PIA in WT brain slices. The amplitude of fEPSP slope was normalized to its baseline amplitude.

FIG. 2C shows a graphical representation of the statistical inhibitory effect of fEPSP slope by 10 nM, 30 nM and 100 nM R-PIA on slices from WT mice.

FIG. 2D shows representative traces of fEPSP recorded during a 180-ms (at bar) in slices from WT, neurabin deficient (Ppp1r9a^{-/-}) and RGS4 deficient (Rgs4^{-/-}) mice.

FIG. 2E shows a representative summary plot of mouse hippocampal slices exposed to 10 nM R-PIA (min 20-50) with a larger magnitude depression in Ppp1r9a^{-/-} and Rgs4^{-/-} mice than in WT mice. The amplitude of fEPSP slope was normalized to its baseline amplitude. N=4/group.

FIG. 2F shows a graphical representation of inhibition of 10 nM R-PIA on slices from WT, Ppp1r9a^{-/-} and Rgs4^{-/-} mice, respectively. ** represents p<0.01 and *** represents p<0.005 by one-way ANOVA multiple comparisons. N=4/group. Data were shown as mean±SEM.

FIG. 3A shows representative fluorescence images of live (top) and dead (bottom) neurons indicating that PI3K/Akt signaling is required for A1R-mediated neuroprotection against glutamate excitotoxicity. WT hippocampal neurons (13-14 DIV) were treated under conditions indicated in FIG. 3B.

FIG. 3B shows a graphical representation that quantifies cell survival and indicates that PI3K/Akt signaling is required for A1R-mediated neuroprotection against glutamate excitotoxicity. WT hippocampal neurons (13-14 DIV) were treated under the indicated conditions. *** represents p<0.001 and **** represents p<0.0001 by one-way ANOVA for multiple comparisons. Sample numbers are indicated in parentheses.

FIG. 4A shows representative Western blots indicating that neurabin deficiency enhances A1R-mediated Akt activation in neurons. Primary hippocampal neurons derived from WT or neurabin deficiency (Ppp1r9a^{-/-}) mice were treated with R-PIA at the indicated concentrations. Representative blots of phospho-Akt (pAkt, Thr308) are shown.

FIG. 4B shows a graphical representation indicating that neurabin deficiency enhances A1R-mediated Akt activation in neurons. Primary hippocampal neurons derived from WT or neurabin deficiency (Ppp1r9a^{-/-}) mice were treated with R-PIA at the indicated concentrations. Quantitation of phospho-Akt (pAkt, Thr308) is shown. *** represents p<0.001 by two-way ANOVA. N=4/group.

FIG. 4C shows representative Western blots indicating that neurabin deficiency prolongs the A1R-mediated Akt response in neurons. WT and Ppp1r9a^{-/-} neurons were stimulated with 1 μM R-PIA for the indicated time durations. Representative blots of pAkt are shown.

FIG. 4D shows a graphical representation indicating that neurabin deficiency prolongs the A1R-mediated Akt response in neurons. WT and Ppp1r9a^{-/-} neurons were stimulated with 1 μM R-PIA for the indicated time durations. Quantitation of pAkt is shown. * represents p<0.05; ** represents p<0.01; **** represents p<0.0001 by two-way ANOVA for multiple comparisons. N=4/group. Data are shown as mean±SEM.

FIG. 5A is a competition pull-down assay testing the abilities of A1R 3i loop (3iL) and C-tail (CT) peptides in blocking the interaction between GST-Nrb331-453 and [³⁵S]-labeled A1R202-326. Comparable amounts of GST and GST-Nrb331-453 in each reaction were confirmed by Coomassie staining (bottom). A representative autoradiograph (top) shows the amount of [³⁵S] A1R202-326 pulled down by GST-Nrb331-453. Free probe represents 1/10 of the input in each reaction.

FIG. 5B is a competition pull-down assay testing the abilities of A1R 3i loop (3iL) and C-tail (CT) peptides in blocking the interaction between GST-Nrb331-453 and [³⁵S]-labeled A1R202-326. In the graphical representation, quantitation of the amount of [³⁵S] A1R202-326 pulled down by GST-Nrb331-453 is shown. Free probe represents 1/10 of the input in each reaction. * represents p<0.05 vs. BSA control by one-way ANOVA Dunnett's multiple comparisons test. N=3/group.

FIG. 5C shows that expression of GFP tagged A1R-CT (GFP-CT) abolished agonist-induced interaction between neurabin and A1R in intact cells. Representative Western blots are shown.

FIG. 5D shows that expression of GFP tagged A1R-CT (GFP-CT) abolished agonist-induced interaction between neurabin and A1R in intact cells. The graphical representation shows quantitation of the change of neurabin in the IP complex with HA-A1R. For R-PIA vs. vehicle, ** represents p<0.05 and ns represents no significance by two-way ANOVA Sidak's multiple comparisons test. N=3/group.

FIG. 5E shows a graphical representation indicating that the expression of GFP-CT restored A1R-mediated inhibition of cAMP production in the presence of neurabin expression. Cells stably expressing HA-A1R were co-transfected with vectors encoding indicated proteins. Vec represents empty vector. Cells were treated with 10 UM forskolin alone or forskolin plus 1 μM R-PIA. Data are expressed as fold change in cAMP production over forskolin alone control. For forskolin+R-PIA vs. forskolin, *** represents p<0.001 and **** represents p<0.0001 by two-way ANOVA Sidak's multiple comparisons test. N=6 for forskolin and N=7 for forskolin+R-PIA.

FIG. 5F shows a graphical representation indicating that the expression of GFP-CT does not alter the cell surface level of A1R. N=4/group.

FIG. 5G shows representative Western blots indicating that the expression of GFP-CT enhances A1R-mediated Akt activation. Cells stably expressing HA-A1R with GFP or GFP-CT were stimulated with vehicle or 1 μM R-PIA.

FIG. 5H shows a graphical representation indicating that the expression of GFP-CT enhances A1R-mediated Akt activation. Cells stably expressing HA-A1R with GFP or GFP-CT were stimulated with vehicle or 1 μM R-PIA. ** represents p<0.01 by Student's t-test. N=4/group. Error bars represent mean±SEM.

FIG. 6A shows a representative schematic diagram that shows TAT-fused A1R-CT peptide or TAT control peptide infused into the left ventricle 30 min prior to kainate injection.

FIG. 6B shows a graphical representation of seizure severity in response to kainite measured in mice with indicated treatment. For A1R-CT vs TAT, **** represents p<0.0001, and for A1R-CT vs. A1R-CT+DPCPX, #### represents p<0.0001 by two-way ANOVA. N=7 for TAT and A1R-CT+DPCPX; N=8 for A1R-CT.

FIG. 6C shows a graphical representation of maximum seizure scores in mice with indicated treatment. ** represents p<0.01 and *** represents p<0.001 by one-way ANOVA Tukey's multiple comparisons test.

FIG. 6D shows a graphical representation of kainate-induced lethality recorded in mice examined in FIG. 6B. Data are expressed as percentage of death.

FIG. 6E shows representative fluorescence images of cell death in the hippocampal CA1 region as revealed by Fluoro-Jade B (FJB) staining. Scale bar: 100 μm.

FIG. 6F shows a graphical representation of CA1 neurons with positive FJB staining. Data are expressed as the percentage of the level in TAT-treated mice (which is set as 100%). *** represents p<0.001 by paired Student's t test. N=19 slices from 3 mice for TAT and N=19 slices from 3 mice for A1R-CT. Data are expressed as mean±SEM.

FIG. 7A shows a representative schematic diagram of the A1R-CT peptide (fused with TAT) or TAT control peptide administered into the nasal cavity.

FIG. 7B shows a graphical representation of seizure severity in response to kainite measured in mice with the indicated treatment. For A1R-CT vs. TAT, ** represents p<0.01 and *** represents p<0.001, and for A1R-CT vs. A1R-CT+DPCPX, ### represents p<0.0001 by two-way ANOVA Tukey's multiple comparisons test. N=7 for TAT and A1R-CT; N=6 for A1R-CT+DPCPX.

FIG. 7C shows a graphical representation of maximum seizure scores in mice with the indicated treatment. For A1R-CT vs. TAT, *** represents p<0.001, and for A1R-CT vs. A1R-CT+DPCPX, **** represents p<0.0001 by one-way ANOVA Tukey's multiple comparisons test.

FIG. 7D shows a graphical representation of kainate-induced lethality recorded in mice examined in FIG. 7B. Data are expressed as percentage of death.

FIG. 7E shows representative fluorescence images of cell death in the hippocampal CA1 region as revealed by Fluoro-Jade B (FJB) staining. Scale bar: 100 μm.

FIG. 7F shows a graphical representation of CA1 neurons with positive FJB staining. Data are expressed as the percentage of the level in TAT-treated mice (which is set as 100%). *** represents p<0.001 by paired Student's t test. N=15 slices from 3 mice for TAT and N=15 slices from 3 mice for A1R-CT. Data are mean±SEM.

FIG. 8A shows a representative EEG trace and spectrogram in nTg and APP/PS1 mice at baseline.

FIG. 8B shows a representative EEG trace and spectrogram in APP/PS1 mice after TAT treatment.

FIG. 8C shows a representative EEG trace and spectrogram in APP/PS1 mice after TAT-fused A1R-CT peptide treatment.

FIG. 9A shows a representative EEG trace and spectrogram in APP/PS1 mice after A1R-CT+DPCPX treatment.

FIG. 9B shows a graphical representation of spike frequency with repeated measurement in nTg and APP/PS1 mice at the baseline. Data were obtained from analyzing EEG recordings of 3 nTg and 3 APP/PS1 mice. *** represents p<0.001 by a repeated measure ANOVA used to calculate the statistical difference of the spike count per 10 minutes.

FIG. 9C shows a graphical representation of spike frequency with repeated measurement in APP/PS1 mice with indicated treatments. Data were obtained from analyzing EEG recordings of 4 TAT-treated, 4 A1R-CT-treated and 5 A1R-CT+DPCPX-treated APP/PS1 mice. Box-and-whisker plots represent median and 5-95 percentile range of all measurements for each group. ** represents p<0.01, and *** represents p<0.001 by a repeated measure ANOVA used to calculate the statistical difference of the spike count per 10 minutes between nTg and APP/PS1 mice and among different treatments in APP/PS1 mice.

### DETAILED DESCRIPTION

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1% of a given value or range of values. Numerical quantities given in this description are approximate unless stated otherwise, meaning that the term “about” or “approximately” can be inferred when not expressly stated.

The terminology used herein is for the purpose of describing particular embodiments only and is not intended to be limiting. As used herein, the singular forms “a”, “an” and “the” are intended to include the plural forms as well, unless the context clearly indicates otherwise.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Terms such as “at least one of A and B” should be understood to mean “only A, only B, or both A and B.” The same construction should be applied to longer list (e.g., “at least one of A, B, and C”).

The term “consisting essentially of” means that, in addition to the recited elements, what is claimed may also contain other elements (steps, structures, ingredients, components, etc.) that do not adversely affect the operability of what is claimed for its intended purpose as stated in this disclosure. This term excludes such other elements that adversely affect the operability of what is claimed for its intended purpose as stated in this disclosure, even if such other elements might enhance the operability of what is claimed for some other purpose.

In this disclosure terms such as “administering” or “administration” include acts such as prescribing, dispensing, giving, or taking a substance such that what is prescribed, dispensed, given, or taken is actually contacts the patient's body externally or internally (or both). It is specifically contemplated that instructions or a prescription by a medical professional to a subject or patient to take or otherwise self-administer a substance is an act of administration.

The term “therapeutically effective amount” as used herein refers to an amount of a compound, either alone or as a part of a pharmaceutical composition, that is capable of having any detectable, positive effect on any symptom, aspect, or characteristics of a disease state or condition. Such effect need not be absolute to be beneficial.

The term “prodrug” as used herein includes functional derivatives of a disclosed compound which are readily convertible in vivo into the required compound. Thus, in the methods of treatment of the present disclosure, the term “administering” shall encompass the treatment of the various disease states/conditions described with the compound specifically disclosed or with a prodrug which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “pharmaceutically acceptable salts” as used herein includes salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “nucleotide” as used herein refer to any such known groups, natural or synthetic. It includes conventional DNA or RNA bases (A, G, C, T, U), base analogs (e.g., inosine, 5-nitroindazole and others), imidazole-4-carboxamide, pyrimidine or purine derivatives (e.g., modified pyrimidine base 6H,8H-3,4-dihydropyrimido[4,5-c][1,2]oxazin-7-one (sometimes designated “P” base that binds A or G)) and modified purine base N6-methoxy-2,6-diaminopurine (sometimes designated “K” base that binds C or T), hypoxanthine, N-4-methyl deoxyguanosine, 4-ethyl-2′-deoxycytidine, 4,6-difluorobenzimidazole and 2,4-difluorobenzene nucleoside analogues, pyrene-functionalized LNA nucleoside analogues, deaza- or aza-modified purines and pyrimidines, pyrimidines with substituents at the 5 or 6 position and purines with substituents at the 2, 6 or 8 positions, 2-aminoadenine (nA), 2-thiouracil (sU), 2-amino-6-methylaminopurine, O-6-methylguanine, 4-thio-pyrimidines, 4-amino-pyrimidines, 4-dimethylhydrazine-pyrimidines, O-4-alkyl-pyrimidines and hydrophobic nucleobases that form duplex DNA without hydrogen bonding. Nucleobases can be joined together by a variety of linkages or conformations, including phosphodiester, phosphorothioate or methylphosphonate linkages, peptide-nucleic acid linkages.

The term “polynucleotide” as used herein refers to a multimeric compound comprising nucleotides linked together to form a polymer, including conventional RNA, DNA, LNA, BNA, copolymers of any of the foregoing, and analogs thereof.

Nucleic acids are “complementary” to each other, as used herein, when a nucleotide sequence in one strand of a nucleic acid, due to orientation of its nucleotide hydrogen atoms, hydrogen bonds to another sequence on an opposing nucleic acid strand (of course, a strand of a nucleic acid may be self-complementary as well). The complementary bases typically are, in DNA, A with T, and C with G, and, in RNA, C with G, and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not perfectly complementary to a sequence in an opposing strand, but that sufficient bonding occurs between bases on the two strands to form a stable hybrid complex at a given set of hybridization conditions (e.g., salt concentration and temperature). Such conditions can be predicted by using the sequences and standard models to predict the Tₘ of hybridized strands, or by empirical determination of Tₘ by using established methods. Tₘ refers to the temperature at which a population of hybridization complexes formed between two nucleic acid strands are 50% denatured. At a temperature below the Tₘ, formation of a hybridization complex is favored, whereas at a temperature above the Tm, melting or separation of the strands in the hybridization complex is favored.

The term “nucleic acid” as used herein refers to a single stranded polynucleotide or a duplex of two polynucleotides. Such duplexes need not be annealed at all locations, and may contain gaps or overhangs.

The term “genetically modified” means that genetic material has been altered by human intervention. In the context of a self-replicating entity such as a cell or a virus, such alteration may have been performed on the self-replicating entity in question, or on an ancestor of the self-replicating entity from whom it acquired the alteration.

### B. Anticonvulsant/Neuroprotective Agents

The present disclosure provides for compounds that increase A1R activity, either directly or through inhibition of expression, either in vitro or in vivo. The present disclosure also provides for compounds that inhibit or reduce the ability of neurabin to bind to A1R, at least insofar as such binding results in a reduction in A1R activity. Throughout the brain, activation of A1R is believed to limit neuronal excitability through coupling to K⁺ channels causing hyperpolarization and indirectly by decreasing glutamate release. Additionally, A1R is believed to be a critical player mediating the negative feedback control of neuronal activity by microglia. Insofar as necessary to clarify what is meant by “activity” of A1R, such activity may include at least: activity to limit neuronal excitability, mediation of negative feedback control of neuronal activity by microglia, or both.

Such compounds are referred to herein as “active compounds” or “active agents.”

Unless stated otherwise, all active compounds are to be construed as including a metabolite; such a metabolite may be formed either in vivo within the body or as a result of biochemical activity in vitro. It is within the scope of this disclosure than any active compound may be limited to a non-metabolite compound.

A1R is a protein receptor for adenosine present in many species, and relatively conserved among mammals. The activity of this receptor is mediated by G proteins which inhibit adenylyl cyclase. Multiple domains of A1R bind to neurabin, including at the C-terminal domain and the 3i loop. Numerous canonical amino acid sequences of A1R have been elucidated, including in human (Uniprot entry P30542—SEQ ID NO: 1), orangutan (Uniprot entry Q5RF57—SEQ ID NO:2), rat (Uniprot entry P25099—SEQ ID NO:3), mouse (Uniprot entry Q60612—SEQ ID NO:4), cavy (Uniprot entry P47745—SEQ ID NO:5), dog (Uniprot entry P11616—SEQ ID NO:6), and cattle (Uniprot entry P28190—SEQ ID NO:7). The amino acid sequences in the foregoing Uniprot entries are incorporated herein by reference as necessary to define and describe A1R in these species. Examples of canonical A1R peptide sequences and a consensus sequence are shown in Table 1, wherein “SID” refers to SEQ ID NO.

TABLE 1

| | | |
|---|---|---|
| SP\|Q5RF57\|SID2_PONAB | MPPSISAFQAAYIGIEVLIALVSVPGNVLVIWAVKVNQALRDATFCFIVLLAVADVAVGA | 60 |
| SP\|P47745\|SID5_CAVPO | MPHSVSAFQAAYIGIEVLIALVSVPGNVLVIWAVKVNQALRDATFCFIASLAVADVAVGA | 60 |
| SP\|P25099\|SID3_RAT | MPPYISAFQAAYIGIEVLIALVSVPGNVLVIWAVKVNQALRDATFCFIVSLAVADVAVGA | 60 |
| SP\|P28190\|SID7_BOVIN | MPPSISAFQAAYIGIEVLIALVSVPGNVLVIWAVKVNQALRDATFCFIVSLAVADVAVGA | 60 |
| SP\|P30542\|SID1_HUMAN | MPPSISAFQAAYIGIEVLIALVSVPGNVLVIWAVKVNQALRDATFCFIVSLAVADVAVGA | 60 |
| SP\|P11616\|SID6_CANLF | MPPAISAFQAAYIGIEVLIALVSVPGNVLVIWAVKVNQALRDATFCFIVSLAVADVAVGA | 60 |
| SP\|Q60612\|SID4_MOUSE | MPPYISAFQAAYIGIEVLIALVSVPGNVLVIWAVKVNQALRDATFCFIVSLAVADVAVGA | 60 |
| CONSENSUS\|SID12 | ** :*******************************************. ********** | |
| SP\|Q5RF57\|SID2_PONAB | LVIPLAILINIGPQTYFHTCLMVACPVLILTQSSILALLTIAVDRYLRVKIPLRYKMVVT | 120 |
| SP\|P47745\|SID5_CAVPO | LVIPLAILINIGPQTYFHTCLMVACPVLILTQSSILALLAIAVDRYLRVKIPLRYKTVVT | 120 |
| SP\|P25099\|SID3_RAT | LVIPLAILINIGPQTYFHTCLMVACPVLILTQSSILALLAIAVDRYLRVKIPLRYKTVVT | 120 |
| SP\|P28190\|SID7_BOVIN | LVIPLAILINIGPRTYFHTCLKVACPVLILTQSSILALLAIAVDRYLRVKIPLRYKTVVT | 120 |
| SP\|P30542\|SID1_HUMAN | LVIPLAILINIGPQTYFHTCLMVACPVLILTQSSILALLAIAVDRYLRVKIPLRYKMVVT | 120 |
| SP\|P11616\|SID6_CANLF | LVIPLAILINIGPRTYFHTCLMVACPVLILTQSSILALLAIAVDRYLRVKIPLRYKTVVT | 120 |
| SP\|Q60612\|SID4_MOUSE | LVIPLAILINIGPQTYFHTCLMVACPVLILTQSSILALLAIAVDRYLRVKIPLRYKTVVT | 120 |
| CONSENSUS\|SID12 | *************:******* *****************:**************** *** | |
| SP\|Q5RF57\|SID2_PONAB | PRRAAVAIAGCWILSFVVGLTPMFGWNNLSAVERAWAANGSMGEPVIKCEFEKVISMEYM | 180 |
| SP\|P47745\|SID5_CAVPO | PRRAAVAIAGCWILSLVVGLTPMFGWNNLSKIEMAWAANGSVGEPVIKCEFEKVISMEYM | 180 |
| SP\|P25099\|SID3_RAT | QRRAAVAIAGCWILSLVVGLTPMFGWNNLSVVEQDWRANGSVGEPVIKCEFEKVISMEYM | 180 |
| SP\|P28190\|SID7_BOVIN | PRRAVVAITGCWILSFVVGLTPMFGWNNLSAVERDWLANGSVGEPVIECQFEKVISMEYM | 180 |
| SP\|P30542\|SID1_HUMAN | PRRAAVAIAGCWILSFVVGLTPMFGWNNLSAVERAWAANGSMGEPVIKCEFEKVISMEYM | 180 |
| SP\|P11616\|SID6_CANLF | PRRAAVAIAGCWILSFVVGLTPLFGWNRLGEAQRAWAANGSGGEPVIKCEFEKVISMEYM | 180 |
| SP\|Q60612\|SID4_MOUSE | QRRAAVAIAGCWILSLVVGLTPMFGWNNLSEVEQAWIANGSVGEPVIKCEFEKVISMEYM | 180 |
| CONSENSUS\|SID12 | ***.***:******:******:****.*. : * **** *****:*:********** | |
| SP\|Q5RF57\|SID2_PONAB | VYFNFFVWVLPPLLLMVLIYLEVFYLIRKQLNKKVSASSGDPQKYYGKELKIAKSLALIL | 240 |
| SP\|P47745\|SID5_CAVPO | VYFNFFVWVLPPLLLMVLIYLEVFYLIRKQLSKKVSASSGDPQKYYGKELKIAKSLALIL | 240 |
| SP\|P25099\|SID3_RAT | VYFNFFVWVLPPLLLMVLIYLEVFYLIRKQLNKKVSASSGDPQKYYGKELKIAKSLALIL | 240 |
| SP\|P28190\|SID7_BOVIN | VYFNFFVWVLPPLLLMVLIYMEVFYLIRKQLNKKVSASSGDPQKYYGKELKIAKSLALIL | 240 |
| SP\|P30542\|SID1_HUMAN | VYFNFFVWVLPPLLLMVLIYLEVFYLIRKQLNKKVSASSGDPQKYYGKELKIAKSLALIL | 240 |
| SP\|P11616\|SID6_CANLF | VYFNFFVWVLPPLLLMVLIYLEVFYLIRRQLGKKVSASSGDPQKYYGKELKIAKSLALIL | 240 |
| SP\|Q60612\|SID4_MOUSE | VYFNFFVWVLPPLLLMVLIYLEVFYLIRKQLNKKVSASSGDPQKYYGKELKIAKSLALIL | 240 |
| CONSENSUS\|SID12 | ********************:*******:**.**************************** | |
| SP\|Q5RF57\|SID2_PONAB | FLFALSWLPLHILNCITLFCPSCHKPSILTYIAIFLTHGNSAMNPIVYAFRIQKFRVTFL | 300 |
| SP\|P47745\|SID5_CAVPO | FLFALSWLPLHILNCITLFCPTCHKPTILTYIAIFLTHGNSAMNPIVYAFRIQKFRVTFL | 300 |
| SP\|P25099\|SID3_RAT | FLFALSWLPLHILNCITLFCPTCQKPSILIYIAIFLTHGNSAMNPIVYAFRIHKFRVTFL | 300 |
| SP\|P28190\|SID7_BOVIN | FLFALSWLPLHILNCITLFCPSCHMPRILIYIAIFLSHGNSAMNPIVYAFRIQKFRVTFL | 300 |
| SP\|P30542\|SID1_HUMAN | FLFALSWLPLHILNCITLFCPSCHKPSILTYIAIFLTHGNSAMNPIVYAFRIQKFRVTFL | 300 |
| SP\|P11616\|SID6_CANLF | FLFALSWLPLHILNCITLFCPSCRKPSILMYIAIFLTHGNSAMNPIVYAFRIQKFRVTFL | 300 |
| SP\|Q60612\|SID4_MOUSE | FLFALSWLPLHILNCITLFCPTCQKPSILIYIAIFLTHGNSAMNPIVYAFRIHKFRVTFL | 300 |
| CONSENSUS\|SID12 | *********************:*: * ** ******:***************:******* | |
| SP\|Q5RF57\|SID2_PONAB | KIWNDNFRCQPAPPIDEDLPEERPDD | 326 |
| SP\|P47745\|SID5_CAVPO | KIWNDHFRCQPEPPIDEDLPEEKVDD | 326 |
| SP\|P25099\|SID3_RAT | KIWNDHFRCQPKPPIDEDLPEEKAED | 326 |
| SP\|P28190\|SID7_BOVIN | KIWNDHFRCQPAPPVDEDAPAERPDD | 326 |
| SP\|P30542\|SID1_HUMAN | KIWNDHFRCQPAPPIDEDLPEERPDD | 326 |
| SP\|P11616\|SID6_CANLF | KIWNDHFRCQPTPPVDEDPPEEAPHD | 326 |
| SP\|Q60612\|SID4_MOUSE | KIWNDHFRCQPKPPIEEDIPEEKADD | 326 |
| CONSENSUS\|SID12 | *****:***** **::** * * .* | |

Natural variants have been noted at positions 43 (A->S), 50 (S->P), 105 (R->H), 170 (E->K), 261 (P->Q), and 279 (G->S) in the canonical human sequence. Some embodiments of the A1R polypeptide comprises one or more of the foregoing substitutions.

It has been discovered that the C-terminal region of A1R, but not the 3i region, is an anticonvulsant and neuroprotectant. A medicament for the treatment or prevention of a neurological condition comprising a therapeutically effective amount of an agent comprising a peptide sequence from the C-terminal region of A1R or a functional derivative thereof is provided. This is referred to herein a “the A1R tail sequence,” or “A1R-CT.”

The agent may also contain a cell-permeable transporter tag, to facilitate the importation of the agent into the cytoplasm, which is where A1R and neurabin interact. Some embodiments of the cell-permeable transporter tag comprise a peptide sequence that facilitates transport through the cell membrane. Some embodiments of the agent comprise a transporter protein disclosed in the Transporter Classification Database, available at https://tcdb.org/. Some embodiments of the transporter tag are the trans-activator of transcription (TAT) protein of HIV-1 or a functional derivative thereof.

The agent may comprise a linker group between the cell-permeable transporter tag and the C-terminal region of the A1R. The linker group functions to distance the A1R-CT group from the transport tag, which could be necessary to allow one or both of those groups to associate with their ligands unhindered. Some embodiments of the linker group are a peptide group. In a specific embodiment the linker group is a series of GS repeats. Some embodiments of the linker have 6 residues. Further embodiments may comprise 3-9 residues, 4-8 residues, and 5-7 residues. In a specific embodiment the linker is GSGSGS.

The peptide sequence from the A1R-CT may contain about 36 amino acid residues. Some embodiments of the peptide sequence from the A1R-CT contain up to 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 residues, or about any of the foregoing. Further embodiments of the peptide sequence from the A1R-CT contain at least 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 residues, or about any of the foregoing. Some embodiments of the peptide sequence from the A1R-CT contain about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 residues. A specific embodiment of the A1R-CT contains 36-44 residues. A more specific embodiment of the A1R-CT contains 44 residues.

The peptide sequence from the A1R-CT may be some or all of the C-terminal region of the human A1R sequence of SEQ ID NO: 1, mouse sequence of SEQ ID NO: 4, consensus sequence SEQ ID NO: 12, or a functional derivative any of the foregoing. In further embodiments the A1R-CT peptide sequence is some or all of the C-terminal region of any of SEQ ID NOS: 2, 3, and 5-7 or a functional derivative of any of the foregoing. Some embodiments of the peptide sequence comprise some or all of the 36 C-terminal amino acids in any of the A1R sequences disclosed above.

The present disclosure contemplates the use of A1R-CT derivatives in the methods of treatment and prevention disclosed herein. An A1R-CT derivative as defined herein refers to an A1R-CT polypeptide that includes a one or more fragments, insertions, deletions or substitutions. The A1R-CT derivative may have an activity that is comparable to or increased (in one embodiment, 50% or more) as compared to the wild-type A1R-CT activity and as such may be used to increase a A1R-CT activity; alternatively, the A1R-CT derivative may have an activity that is decreased (in one embodiment, less than 50%) as compared to the wild-type A1R-CT activity and as such may be used to decrease a A1R-CT activity. In some cases the derivative will retain antigenic specificity of A1R-CT.

A fragment of A1R-CT is any polypeptide consisting of any number of adjacent amino acid residues having the same identity and order as any segment of A1R-CT. Conservative modifications to the amino acid sequence of any fragment are also included (conservative substitutions are discussed below). Such fragments can be produced for example by digestion of A1R-CT with an endoprotease (which will produce two or more fragments) or an exoprotease. A fragment may be of any length up to the length of A1R-CT. A fragment may be, for example, at least 3 residues in length. A fragment that is at least 6 residues in length will generally function as an antigenic group. Such groups would be expected to be cross-recognized by some antibodies specific for A1R-CT.

Derivatives of A1R-CT will have some degree of identity with native A1R-CT. For example, it would be expected that most derivatives having from 95-100% identity with native A1R-CT would retain the function of A1R-CT. There is also a likelihood that functionality would be retained by a homolog to A1R-CT within any one of the following ranges of identity: 75-100%, 80-100%, 85-100%, 90-100%, 96-100%, 97-100%, 98-100% and 99-100%. The minimum desirable identity can be determined in some cases by identifying a known non-functional homolog to A1R-CT, and establishing that the minimum desirable identity must be above the identity between A1R-CT and the known non-functional identity. The minimum desirable identity can be determined in some cases by identifying a known functional homolog to A1R-CT, and establishing that the range of desirable identity must encompass the percent identity between A1R-CT and the known non-functional identity.

The deletions, additions and substitutions can be selected to generate a desired A1R-CT derivative. Likewise conservative substitutions or substitutions of amino acids with similar properties is expected to be tolerated and A1R-CT activity may be conserved. In addition, specific deletions, insertions and substitutions may impact, positively or negatively, a certain A1R-CT activity but not impact another A1R-CT activity.

Conservative modifications to the amino acid sequence of the A1R-CT (and the corresponding modifications to the encoding nucleotides) will produce A1R-CT derivatives having functional and chemical characteristics similar to those of naturally occurring A1R-CT. In contrast, substantial modifications in the functional and/or chemical characteristics of A1R-CT may be accomplished by selecting substitutions in the amino acid sequence of that differ significantly in their effect on maintaining (a) the structure of the molecular backbone in the area of the substitution, (b) the charge or hydrophobicity of the molecule at the binding site for a binding target, or (c) the bulk of a side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine.

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties. Nucleic acid and polypeptide molecules described herein may be chemically synthesized as well as produced by recombinant means.

Naturally occurring residues may be divided into classes based on common side chain properties: 1) hydrophobic: norleucine, Met, Ala, Val, Leu, Ile; 2) neutral hydrophilic: Cys, Ser, Thr, Asn, Gln; 3) acidic: Asp, Glu; 4) basic: His, Lys, Arg; 5) residues that influence chain orientation: Gly, Pro; and 6) aromatic: Trp, Tyr, Phe.

For example, non-conservative substitutions may involve the exchange of a member of one of these classes for a member from another class.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art (Kyte et al., J. Mol. Biol., 157:105-131, 1982). It is known that certain amino acids may be substituted for other amino acids having a similar hydropathic index or score and still retain a similar biological activity.

In making changes based upon the hydropathic index, the substitution of amino acids whose hydropathic indices are within +/−2 may be used; in an alternate embodiment, the hydropathic indices are with +/−1; in yet another alternate embodiment, the hydropathic indices are within +/−0.5.

It is also understood in the art that the substitution of like amino acids can be made effectively on the basis of hydrophilicity. The greatest local average hydrophilicity of a polypeptide as governed by the hydrophilicity of its adjacent amino acids, correlates with a biological property of the protein.

The following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within +/−2 may be used; in an alternate embodiment, the hydrophilicity values are with +/−1; in yet another alternate embodiment, the hydrophilicity values are within +/−0.5.

Desired amino acid substitutions (whether conservative or non-conservative) can be determined at the time such substitutions are desired. For example, amino acid substitutions can be used to identify important residues of the A1R-CT, or to increase or decrease the affinity of the A1R-CT with a particular binding target in order to increase or decrease A1R-CT activity. Exemplary amino acid substitutions are set forth in Table 2.

TABLE 2

| Original | | Preferred |
|---|---|---|
| Amino Acid | Exemplary substitution | substitution |
| Ala | Val, Leu, Ile | Val |
| Arg | Lys, Gln, Asn | Lys |
| Asn | Glu | Glu |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Ile, Val, Met, Ala, Phe, Norleucine | Ile |
| Lys | Arg, 1,4-diaminobutyric acid, Gln, Asn | Arg |
| Met | Leu, Phe, Ile | Leu |
| Phe | Leu, Val, Ile, Ala, Tyr | Leu |
| Pro | Ala, Gly | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Suitable variants of the polypeptide as set forth in any of SEQ ID NOS: 1-7, and 10-12 can be determined, including combinations thereof, using various techniques. For identifying suitable areas of the molecule that may be changed without destroying activity, one may target areas not believed to be important for activity. For example, when similar polypeptides with similar activities from the same species or from other species are known, one may compare the amino acid sequence of an A1R-CT to such similar polypeptides. With such a comparison, one can identify residues and portions of the molecules that are conserved among similar polypeptides. It will be appreciated that changes in areas of a A1R-CT that are not conserved relative to such similar polypeptides would be less likely to adversely affect the biological activity and/or structure of the A1R-CT. Even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one can review structure-function studies identifying residues in similar polypeptides that are important for activity or structure. In view of such a comparison, one can predict the importance of amino acid residues in A1R-CT that correspond to amino acid residues that are important for activity or structure in similar polypeptides. One may opt for chemically similar amino acid substitutions for such predicted important amino acid residues of A1R-CT.

One can also analyze the three-dimensional structure and amino acid sequence in relation to that structure in similar polypeptides. In view of that information, one may predict the alignment of amino acid residues of A1R-CT with respect to its three dimensional structure. One may choose not to make radical changes to amino acid residues predicted to be on the surface of the protein, since such residues may be involved in important interactions with other molecules. Moreover, one may generate test A1R-CT derivatives containing a single amino acid substitution at each desired amino acid residue. The derivatives can then be screened using activity assays, including those disclosed herein. Such derivatives could be used to gather information about suitable substitution. For example, if one discovered that a change to a particular amino acid residue resulted in destroyed, undesirably reduced, or unsuitable activity, derivatives with such a change would be avoided. In other words, based on information gathered from such routine experiments, one can determine the amino acids where further substitutions should be avoided either alone or in combination with other mutations.

Numerous scientific publications have been devoted to the prediction of secondary structure from analyses of amino acid sequences (see Chou et al., Biochemistry, 13 (2): 222-245, 1974; Chou et al., Biochemistry, 113 (2): 211-222, 1974; Chou et al., Adv. Enzymol. Relat. Areas Mol. Biol., 47:45-148, 1978; Chou et al., Ann. Rev. Biochem., 47:251-276, 1979; and Chou et al., Biophys. J., 26:367-384, 1979). Moreover, computer programs are currently available to assist with predicting secondary structure of polypeptides. Examples include those programs based upon the Jameson-Wolf analysis (Jameson et al., Comput. Appl. Biosci., 4 (1): 181-186, 1998; and Wolf et al., Comput. Appl. Biosci., 4 (1): 187-191; 1988), the program PepPlot® (Brutlag et al., CABS, 6:237-245, 1990; and Weinberger et al., Science, 228:740-742, 1985), and other new programs for protein tertiary structure prediction (Fetrow. et al., Biotechnology, 11:479-483, 1993).

Moreover, computer programs are currently available to assist with predicting secondary structure. One method of predicting secondary structure is based upon identity modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure (see Holm et al., Nucl. Acid. Res., 27 (1): 244-247, 1999).

Additional methods of predicting secondary structure include “threading” (Jones, D., Curr. Opin. Struct. Biol., 7 (3): 377-87, 1997; Suppl et al., Structure, 4 (1): 15-9, 1996), “profile analysis” (Bowie et al., Science, 253:164-170, 1991; Gribskov et al., Meth. Enzym., 183:146-159, 1990; and Gribskov et al., Proc. Nat. Acad. Sci., 84 (13): 4355-4358, 1987), and. “evolutionary linkage” (See Home, supra, and Brenner, supra).

Another embodiment of the active agent is a nucleic acid agent. Such nucleic acid agents include a gene therapy agent that causes cells in the subject to express any of the agents disclosed above. The cells expressing the agent can be introduced into the subject, or the cells can be altered to express the agents in situ. In a specific embodiment an mRNA molecule encoding the active agent is introduced to the subject's cells, for example in a vehicle for hypodermic injection. In further embodiments the cell contains a DNA sequence that encodes the active agent, and is introduced to the subject.

### C. Medicaments and Pharmaceutical Compositions

Useful compositions of the present disclosure may comprise one or more active agents as described above. In one embodiment, such compounds are in the form of compositions, such as but not limited to, pharmaceutical compositions and medicaments. The compositions disclosed may comprise one or more of such compounds, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor). To form a pharmaceutically acceptable composition suitable for administration, such compositions will contain a therapeutically effective amount of a compound(s).

The pharmaceutical compositions of the disclosure may be used in the treatment and prevention methods of the present disclosure. Such compositions are administered to a subject in amounts sufficient to deliver a therapeutically effective amount of the compound(s) to be effective in the treatment and prevention methods disclosed herein. The therapeutically effective amount may vary according to a variety of factors such as, but not limited to, the subject's condition, weight, sex and age. Other factors include the mode and site of administration. The pharmaceutical compositions may be provided to the subject in any method known in the art. In one embodiment of the method administration is performed intranasally. Exemplary routes of administration include, but are not limited to, subcutaneous, intravenous, topical, epicutaneous, oral, intraosseous, intramuscular, and pulmonary. The compositions of the present disclosure may be administered only one time to the subject or more than one time to the subject. Furthermore, when the compositions are administered to the subject more than once, a variety of regimens may be used, such as, but not limited to, one per day, once per week, once per month or once per year. The compositions may also be administered to the subject more than one time per day. The therapeutically effective amount of the nucleic acid molecules and appropriate dosing regimens may be identified by routine testing to obtain optimal activity, while minimizing any potential side effects. In addition, co-administration or sequential administration of other agents may be desirable.

The active agents described herein may be administered in a therapeutically effective amount of 1-100 mg/kg. Preferably, the therapeutically effective amount is 1.1-90 mg/kg. More preferably, the therapeutically effective amount is 1.25-80 mg/kg. More preferably, the therapeutically effective amount is 1.43-70 mg/kg. More preferably, the therapeutically effective amount is 1.67-60 mg/kg. More preferably, the therapeutically effective amount is 2-50 mg/kg. More preferably, the therapeutically effective amount is 2.5-40 mg/kg. More preferably, the therapeutically effective amount is 3.33-30 mg/kg. More preferably, the therapeutically effective amount is 5-20 mg/kg. In a preferred embodiment, the therapeutically effective amount is 10 mg/kg or about 10 mg/kg.

The compositions of the present disclosure may be administered systemically, such as by intravenous administration, or locally such as by subcutaneous injection or by application of a paste or cream. Some embodiments of the composition are gene therapy compositions, such as compositions to deliver nucleic acid vectors or mRNA.

The compositions of the present disclosure may further comprise agents which improve the solubility, half-life, absorption, etc. of the compound(s). Furthermore, the compositions of the present disclosure may further comprise agents that attenuate undesirable side effects and/or or decrease the toxicity of the compounds(s). Examples of such agents are described in a variety of texts, such a, but not limited to, Remington: The Science and Practice of Pharmacy (20^{th} Ed., Lippincott, Williams & Wilkins, Daniel Limmer, editor).

The compositions of the present disclosure can be administered in a wide variety of dosage forms for administration. For example, the compositions can be administered in forms, such as, but not limited to, tablets, capsules, sachets, lozenges, troches, pills, powders, granules, elixirs, tinctures, solutions, suspensions, elixirs, syrups, ointments, creams, pastes, emulsions, or solutions for intravenous administration or injection. Other dosage forms include administration transdermally, via patch mechanism or ointment. Further dosage forms include formulations suitable for delivery by nebulizers or metered dose inhalers. Any of the foregoing may be modified to provide for timed release and/or sustained release formulations.

In the present disclosure, the pharmaceutical compositions may further comprise a pharmaceutically acceptable carrier. Such carriers include, but are not limited to, vehicles, adjuvants, surfactants, suspending agents, emulsifying agents, inert fillers, diluents, excipients, wetting agents, binders, lubricants, buffering agents, disintegrating agents and carriers, as well as accessory agents, such as, but not limited to, coloring agents and flavoring agents (collectively referred to herein as a carrier). Typically, the pharmaceutically acceptable carrier is chemically inert to the active compounds and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, for oral administration in solid form, such as but not limited to, tablets, capsules, sachets, lozenges, troches, pills, powders, or granules, the compound(s) may be combined with an oral, non-toxic pharmaceutically acceptable inert carrier, such as, but not limited to, inert fillers, suitable binders, lubricants, disintegrating agents and accessory agents. Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthum gum and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acadia, emulsions, and gels containing, in addition to the active ingredient, such carriers as are known in the art.

For oral liquid forms, such as but not limited to, tinctures, solutions, suspensions, elixirs, syrups, the nucleic acid molecules of the present disclosure can be dissolved in diluents, such as water, saline, or alcohols. Furthermore, the oral liquid forms may comprise suitably flavored suspending or dispersing agents such as the synthetic and natural gums, for example, tragacanth, acacia, methylcellulose and the like. Moreover, when desired or necessary, suitable and coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The compound(s) may be administered in a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as, but not limited to, a soap, an oil or a detergent, suspending agent, such as, but not limited to, pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyldialkylammonium halides, and alkylpyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkylbeta-aminopropionates, and 2-alkylimidazoline quaternary ammonium salts, and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as, but not limited to, ointments, creams, pastes, emulsions, containing the nucleic acid molecule of the present disclosure, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage or dressing for transdermal delivery or may be applied to a bandage or dressing for delivery directly to the site of a wound or cutaneous injury.

The compound(s) of the present disclosure can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. Such liposomes may also contain monoclonal antibodies to direct delivery of the liposome to a particular cell type or group of cell types.

The compound(s) of the present disclosure may also be coupled with soluble polymers as targetable drug carriers. Such polymers can include, but are not limited to, polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

### D. Methods of Treatment and Prevention

The teachings of the present disclosure provide for the treatment and/or prevention of neurological disease states and conditions in a subject in need of such treatment and/or prevention. Neurological disease states and conditions include seizure disorders, such as those caused by neuronal excitability. Such disease states and conditions include, but are limited to, epilepsy, brain injury, stroke, Alzheimer's disease, Parkinson's disease, and Lewy body dementia. Neurological disease states and conditions associated with cell death include those caused by at least one of hypoxia, ischemia, excitotoxin exposure, traumatic injury, intracerebral hemorrhage, chemical agents, and aglycemia.

The method of treatment and/or prevention comprises administering to the subject any of the active compounds disclosed herein. The method will often further comprise identifying a subject in need of such treatment or prevention.

Said treatment and/or prevention is accomplished by disrupting the A1R-neurabin interaction and/or reversing neurabin-mediated attenuation of A1R signaling. Such decreased A1R-neurabin interactions are accomplished by administering an active compound or pharmaceutical composition containing at least one active compound such as but not limited to, a specific or non-specific inhibitor of such polypeptides, agents that reduce the stability or half-life of such polypeptides, or agents that promote the intracellular sequestration of such polypeptides. Any inhibitor known or subsequently determined to disrupt the A1R-neurabin interaction and/or reverse neurabin-mediated attenuation of A1R signaling may be used.

In certain embodiments of the treatment and/or prevention methods disclosed, the results of disrupting the A1R-neurabin interaction and/or reversing neurabin-mediated attenuation of A1R signaling include, but are not limited to increasing the activity of the adenosine A1 receptor (A1R) and reducing binding of the adenosine A1 receptor (A1R) to neurabin.

The compounds/agents used in the above methods may be functional nucleic acids, polypeptides, or compounds identified in the assays disclosed herein.

### E. Nucleic Acids

Nucleic acids are provided that encode any of the active agents described above. Such nucleic acids comprise a sequence encoding any one or more of the active agents. The sequence of the nucleic acid can be determined based on the triplet code by which nucleic acid codons are transcribed (in some cases after translation) into amino acids. These can be based on the standard codons, both DNA and RNA; in some expression systems nonstandard versions of the code may be used. Such nonstandard codons can be useful in some natural organisms that use nonstandard versions of the code and in in vitro expressions systems that use nonstandard versions of the code. In other cases artificially modified versions of the code may be used, for example in organisms that have been modified to use a nonstandard code or in other in vitro expression systems.

Some embodiments of the nucleic acid comprise a promoter operatively linked to the sequence encoding the active agent. In some such embodiments the sequence encoding the active agent will be an open reading frame. In various embodiments the promoter may be a heterologous promoter not naturally linked to a nucleic acid that encodes the active agent.

It is further provided that the nucleic acid is part of a genetically modified cell. The nucleic acid will in some embodiments be in the form of a heterologous gene in the cell. In this context a “heterologous gene” refers to a gene that is not identical to a gene naturally found in the cell. The heterologous gene may be from a different species, or it may be artificial and not found naturally in any species. Generally the presence of a heterologous sequence is the result of genetic modification, and some embodiments of the cell are a genetically modified cell. The cell is considered to be genetically modified if its genetic material has been altered by human intervention; such alteration may have been performed on the cell in question, or on an ancestor of the cell from whom the cell has acquired the heterologous polynucleotide.

The cell may be a cell of a subject to receive treatment or prevention; or a cell of an animal species isolated from the animal (for example in cell culture). Alternatively, the cell may be a unicellular organism or a cell of a multicellular organism. Many unicellular organisms have the advantage of being easier to culture in vitro than cells from multicellular organisms. Unicellular organisms are particularly useful in cloning, replicating, and maintaining nucleic acids of interest. In some embodiments, the cell is a unicellular eukaryotic organism. Unicellular eukaryotic organisms suitable for the method include fungi and protists. Model unicellular organisms that are commonly used for this purpose include yeasts, other fungi, bacteria, protists, and archaea. Specific model organisms are well known in the art, and include bacteria such as Escherichia coli, Salmonella typhimurium, Pseudomonas fluorescens, Bacillus subtilis, Mycoplasma genitalium, and various Synechocystis sp.; protists such as Dictyostelium discoideum, Tetrahymena thermophila, Emiliania huxleyi, and Thalassiosira pseudonana; and fungi such as Aspergillus sp., Neurospora crassa, Saccharomyces cerevisiae, and Schizosaccharomyces pombe.

A vector is also provided, comprising any of the polynucleotides disclosed above, alone or in any combination with one another. Many suitable vectors are known in the art, such as viruses, plasmids, cosmids, fosmids, phagmids, artificial chromosomes, yeast artificial chromosomes, human artificial chromosomes, plant transformation vectors, and liposomes. A specific embodiment of the vector is an expression vector comprising coding regions encoding any of the active agents described above; each said coding region may be operatively linked to a promoter (with the understanding that two or more regions may be linked to the same promoter, so long as each region is linked to a promoter).

### F. Working Examples

The following working examples contain references to journal articles and other sources of information as a means to aid in the understanding and enablement of what is described. Such references are not admissions that any given source of information meets the legal definition of “prior art” in any country; nor are such references admissions that any given source of information is relevant to the patentability of anything that might be claimed; nor are any statements describing what is taught in any given source of information intended to fully characterize what is taught or to fully characterize the relevant teachings of the source of information.

### Introduction

It is demonstrated that neurabin acts as a sensitive rate-limiting factor for modulation of A1R-elicited responses, supporting the feasibility of disruption of the A1R-neurabin interaction as a means to enhance endogenous adenosine-elicited anti-seizure response through A1R. Furthermore, a peptide consisting of the A1R C-terminal sequence (referred to as the A1R-CT peptide) is developed, which disrupts A1R-neurabin interaction and reverses neurabin-mediated attenuation of A1R signaling. This blocking peptide exhibits strong anticonvulsant and neuroprotective effects against kainate-induced seizures when administered through intracerebroventricular or intranasal delivery. Intranasal administration of the A1R-CT peptide also effectively reduces epileptic activities in an AD mouse model. New treatment for epileptic seizures under pathological conditions is possible using the disclosed peptides and compounds.

### Materials and Methods

### Peptides, Antibodies and Chemicals

The TAT alone and TAT-fused A1R-CT peptides were synthesized by GenScript and shown below in Table 3. TAT-fused A1R CT includes the TAT region of GRKKRRQRRR connected to A1R CT by a linker, which has the sequence GSGSGS. The A1R-3iloop and A1R-CT peptide without TAT fusion were synthesized by American Peptide. Phospho-Akt (Thr308) (244F9) Rabbit monoclonal (cat #4056S) and Myc-Tag (9B11) Mouse monoclonal (cat #2276S) antibodies were purchased from Cell Signaling Technology, mouse HA.11 (Biolegend, previously Covance, cat #MMS-101), rat anti-HA from Roche (cat #1815016), and rabbit Neurabin 1 (PPP1R9A) antibody from Epitomics (cat #3717-1). [³⁵S]Methionine was purchased from PerkinElmer. R-PIA [(−)-N6-(2-Phenylisopropyl) adenosine], yohimbine, glutamate, forskolin and LY294002 were from Sigma. Kainite and Fluoro-Jade B were purchased from Milestone Pharm Tech and Fisher. All other chemicals were from Sigma-Aldrich or Fisher.

TABLE 3

| Chimeric Peptide | Sequence (5′-3′) |
|---|---|
| TAT control 1 | GRRRRRRRRR RR |
| (SEQ ID NO: 8) | |
| TAT control 2 | GRKKRRQRRR |
| (SEQ ID NO: 9) | |
| TAT-mA1R | GRKKRRQRRR GSGSGSRIHK FRVTFLKIWN DHFRCQPKPP |
| (SEQ ID NO: 10) | IEEDIPEEKA DD |
| TAT-hA1R | GRKKRRQRRR GSGSGSRIQK FRVTFLKIWN DHFRCQPAPP |
| (SEQ ID NO: 11) | IDEDLPEERP DD |

### Animals

All mice were housed in the AAALAC-accredited Animal Resources Program facility at the University of Alabama at Birmingham in accordance with procedures of the Animal Welfare Act and the 1989 amendments to the Act, and all studies followed protocols approved by the UAB Institutional Animal Care and Use Committee. Generation of the RGS4 deficient (Rgs4^{-/-}) mouse line has been described previously (see, for example, Han M H, Renthal W, Ring R H, Rahman Z, Psifogeorgou K, Howland D, et al. Brain region specific actions of regulator of G protein signaling 4 oppose morphine reward and dependence but promote analgesia. Biological psychiatry. 2010, 67 (8): 761-9). Rgs4^{-/-} mice were compared with their corresponding wild type (WT) mice at the same age. Neurabin deficient (Ppp1r9a^{-/-}) mice were described previously (see, for example, Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95; and Allen P B, Zachariou V, Svenningsson P, Lepore A C, Centonze D, Costa C, et al. Distinct roles for spinophilin and neurabin in dopamine-mediated plasticity. Neuroscience. 2006, 140 (3): 897-911). Neurabin WT, heterozygous, and homozygous littermates were used in this study. APP/PS1 transgenic mice on the C57BL/6 background were described previously (see, for example, Jankowsky J L, Slunt H H, Ratovitski T, Jenkins N A, Copeland N G, and Borchelt D R. Co-expression of multiple transgenes in mouse CNS: a comparison of strategies. BiomolEng. 2001, 17 (6): 157-65; and Chen Y, Peng Y, Che P, Gannon M, Liu Y, Li L, et al. alpha (2A) adrenergic receptor promotes amyloidogenesis through disrupting APP-SorLA interaction. Proc Natl Acad Sci USA. 2014, 111 (48): 17296-301). Ten to eleven month-old gender-matched littermate APP/PS1 and nTg controls were used. Both male and female mice were used, and data were combined, as no significant sex difference was observed. All mice were backcrossed to and maintained on the C57BL/6 genetic background for over 10 generations.

### Induction and Evaluation of Chemoconvulsant Seizures

Induction and evaluation of seizure were performed as described previously (See, for example, Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95). Ten to twelve week-old male mice were injected (i.p.) with kainate (Sigma) at 20 or 25 mg/kg (dissolved in saline), together with or without DPCPX (Sigma, 0.5 mg/kg in saline) or yohimbine (Sigma, 0.5 mg/kg in saline). Seizure severity was scored by trained observers blind to genotype and/or treatment. The severity of seizure behaviors was scored on a scale of 0 to 7, with 0 representing normal behavior and 7 representing death (see, for example, Yang D D, Kuan C Y, Whitmarsh A J, Rincon M, Zheng T S, Davis R J, et al. Absence of excitotoxicity-induced apoptosis in the hippocampus of mice lacking the Jnk3 gene. Nature. 1997, 389 (6653): 865-70; and Cottingham C, Chen Y, Jiao K, and Wang Q. The antidepressant desipramine is an arrestin-biased ligand at the alpha (2A)-adrenergic receptor driving receptor down-regulation in vitro and in vivo. J Biol Chem. 2011, 286 (41): 36063-75). To induce seizures with pentylenetetrazole (PTZ), mice were injected (i.p.) with PTZ at 40 mg/kg (dissolved in saline), with or without 8-Cyclopentyl-1,3-dipropylxanthine (DPCPX). Seizure severity was scored every 4 min for 30 min.

### Acute Hippocampal Slice Preparation and Electrophysiology

Mice (8-12 weeks old) with indicated genotypes were anesthetized with isoflurane. Brains were removed and dissected into coronal slices (350 μm thick) from dorsal hippocampus on a vibratome (VT1000P, Leica Biosystems) using ice-cold high sucrose cutting solution (in mM as follows: 85.0 NaCl, 2.5 KCl, 4.0 MgSO₄, 0.5 CaCl₂, 1.25 NaH₂PO₄, 25.0 glucose, 75.0 sucrose), oxygenated with 95% O₂ and 5% CO₂. After sectioning, slices were transferred to artificial cerebral spinal fluid (ACSF) containing the following (in mM): 119.0 NaCl, 2.5 KCl, 1.3 MgSO₄, 2.5 CaCl₂, 1.0 NaH₂PO₄, 26.0 NaHCO₃, 11.0 glucose. Slices were held from 1 to 5 hours in a submersion chamber continuously bubbled with 95% O₂ and 5% CO₂ at room temperature.

Hippocampal slices were placed in the recording chamber, with continuous perfusion of gassed ACSF at a constant rate (2 mL/min) at room temperature. Extracellular field excitatory postsynaptic potentials (fEPSPs) were recorded in the stratum radiatum using a glass pipette filled with ACSF. A bipolar stimulating electrode was placed in CA1 stratum radiatum to stimulate Schaffer collateral axons (0.1 Hz, 100 μs duration). Baseline field EPSPs (fEPSPs) were obtained at 80-180 μA to elicit baseline fEPSPs of 0.4-0.6 mV in amplitude (40-50% maximal field amplitude) for 20 min. Experiments were excluded if there was more than 8% variance in baseline. Extracellular CA1 pyramidal population spikes (PSs) were evoked using a stimulating electrode positioned in CA1 stratum radiatum to stimulate Schaffer collateral axons at 0.1 Hz with 100 μs duration, and recorded using a glass micropipette placed in stratum pyramidale. Data were collected using an Axopatch 1D amplifier (Molecular Devices, Union City, CA) in current-clamp mode, and signals were filtered at 2 kHz, and acquired using pCLAMP 10.2 acquisition software (Molecular Devices).

### Measurement of CAMP Levels

CAMP assays were performed using AlphaScreen® Assay Kit from PerkinElmer following the manufacturer's instruction. In brief, cultured CHO cells were co-transfected with cDNA encoding HA-A1R, myc-neurabin, and GFP or GFP-A1R293-326. Forty-eight hours post-transfection, cells were washed once and collected in PBS. The pellet was then resuspended with the stimulation buffer (1×HBSS, 0.1% BSA, 0.5 mM IBMX, 5 mM HEPES, pH 7.4) and mixed with anti-cAMP acceptor beads. The mix was divided into 3 groups with the following treatment: (1) vehicle; (2) 10 UM forskolin; (3) 10 UM forskolin and 5 μM R-PIA. Twenty minutes post-stimulation at 37° C., biotinylated cAMP/streptavidin donor beads in lysis buffer (0.1% BSA, 0.3% Tween20, 5 mM HEPES, pH7.4) were added to cells/acceptor beads mix. After 30 min incubation at room temperature, luminescence was analyzed on a Biotek Synergy2 plate reader using standard a-screen settings.

### Intact Cell Surface ELISA

Cell surface HA-A1R expression in CHO-K1 cells transfected with GFP or GFP-A1R-CT was examined by the cell-surface ELISA method as described previously (see, for example, Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95; and Cottingham C, Chen Y, Jiao K, and Wang Q. The antidepressant desipramine is an arrestin-biased ligand at the alpha (2A)-adrenergic receptor driving receptor down-regulation in vitro and in vivo. J Biol Chem. 2011, 286 (41): 36063-75). In brief, cells were fixed, and then subjected to blocking, primary antibody (HA11, 1:3000), and secondary antibody (HRP-conjugated anti-mouse, 1:2000). Following incubation with o-phenylenediamine substrate (Pierce), absorbance at 490 nm was measured to determine surface HA-A1R density.

### In Vitro GST Pull-Down

Preparation of GST fusion proteins, synthesis of [³⁵S]-labeled in vitro translated probes, and pull-down assays were performed as described previously (See, for example, Chen Y, Booth C, Wang H, Wang R X, Terzi D, Zachariou V, et al. Effective Attenuation of Adenosine A1R Signaling by Neurabin Requires Oligomerization of Neurabin. Mol Pharmacol. 2017; 92 (6): 630-9; and Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95). The A1R 3i loop (aa202-235) and C-terminal (aa293-326) peptides used for blocking A1R/neurabin interaction were synthesized at Peptide2.0 Inc. and dissolved in water with the stock concentration at 5 μg/μl. The peptide concentration in final pull-down assay was 0.1 μg/μl.

### Cells and Transfection

CosM6 cells were cultured in DMEM (Life Technologies) supplemented with 10% FBS (Atlanta Biologicals) and 100 U/ml penicillin and 10 μg/mL streptomycin. CHO-K1 cells were cultured in DMEM/F-12 (Invitrogen) with 10% FBS, penicillin and streptomycin, and 2 mM glutamine. Cells were transfected with Lipofectamine 2000 (Life Technologies) following manufacturer's instruction.

### Primary Hippocampal Culture and Glutamate-Induced Cell Death

Primary hippocampal culture was performed as described previously (Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95). To examine the neuroprotective effect of the A1R agonist R-PIA, neuronal death was induced with glutamate under varying conditions and then cell viability was determined using LIVE/DEAD® Viability/Cytotoxicity kit (Life Technologies) following the manufacturer's instruction. In brief, hippocampal neurons were cultured in 24-well plates for 13-14 days in vitro and treated with (1) vehicle, (2) 100 μM glutamate alone, (3) 100 μM glutamate plus 10 μM R-PIA, (4) 100 μM glutamate, 10 μM R-PIA, plus 30 μM LY294002, for 30 min at 37° C. Neurons were then washed twice, and incubated with regular growth medium at 37° C. for an additional 24 hrs. Neurons were stained with 4 μM calcein-AM and 4 μM EthD-1 in DPBS and mounted for imaging under a fluorescence microscope.

### Cannulation and Intracerebroventricular Infusion of Peptide

Stainless-steel single guided cannulas (26 gauge, RWD Life Science, Inc) were implanted into the lateral ventricles with +/−1.0, −0.3, −2.3 (x, y, z) under isoflurane anesthesia, using standard stereotaxic procedures. Coordinates were chosen based on the mouse brain atlas. The cannula was anchored to the skull using screws and acrylic cement. Mice were allowed to recover for 7-10 days after surgery. The injection cannula was connected via PE Tubing (1.50*0.50 mm, RWD Life Science, Inc) to a 10 μL Hamilton micro syringe, driven by a microinjection pump (Dual Syringe, Model ‘11’, Harvard apparatus, MA-70-2209). Infusions were administered in a volume of 5 μL over 10 min, and an additional 1 min was allowed for diffusion before the infusion cannulas were removed. The A1R-CT or TAT control peptide (500 pmole in 5 μL) were administered 30 min prior to intraperitoneal injection of kainate or kainate plus DPCPX.

### Peptide Intranasal Administration

Mice were habituated for a few days (gripping, scruffing and positioning) before peptide administration as described previously (see, for example, Hanson L R, Fine J M, Svitak A L, and Faltesek K A. Intranasal administration of CNS therapeutics to awake mice. J Vis Exp. 2013, (74): 4440). For peptide delivery, mice were held and positioned with the neck and chin flat, and the pipette tip holding the peptide solution was placed at a 45° angle, following a procedure described previously (see, for example, Hanson L R, Fine J M, Svitak A L, and Faltesek K A. Intranasal administration of CNS therapeutics to awake mice. J Vis Exp. 2013, (74): 4440). Six microliter of peptide solution (7.0 mM) was slowly administered to one nostril with 2-3 intervals, and the mouse was held in position for additional 5 sec after the last drop. The administration step was repeated for the other nostril. The total volume injected (12 μL) is lower than that of the mouse nasal cavity which is around 32.5 mm³. Peptides were administered 45 min prior to intraperitoneal injection of kainate. The dosage was estimated to be about 10 mg/kg.

### Assessment of Cell Death in Hippocampal Slices

Seven days after initial kainate exposure, mice were deeply anesthetized with isoflurane and perfused with 4% paraformaldehyde in phosphate-buffered solution, pH 7.4. Hippocampal slices (30 μm) were stained with Fluoro-Jade B staining following the manufacturer's instructions.

### EEG Recordings and Analysis

Implantation of electrodes for EEG recordings was performed under isoflurane anesthesia (4.5% for induction, approximately 2% for maintenance). In brief, 1 mm stainless steel electrodes (P1 Technologies, Roanoke, VA) were implanted into the primary somatosensory cortex at 1.8, 0.02, 1 (AP, ML, DV), and reference electrodes anterior to the bregma were implanted subdurally through small holes drilled in the skull. These electrodes were held in place with Dental cement kit (Stoelting), C&B Metabond Quick Adhesive Cement System (Parkell). The ground electrode was sutured in the cervical paraspinous area. All electrodes were inserted into a six-channel pedestal and connected to the commutator for recording (see, for example, Johnson E C B, Ho K, Yu G-Q, Das M, Sanchez P E, Djukic B, et al. Behavioral and neural network abnormalities in human APP transgenic mice resemble those of App knock-in mice and are modulated by familial Alzheimer's disease mutations but not by inhibition of BACE1. Molecular Neurodegeneration. 2020, 15 (1): 53.). Mice were allowed to recover for 5-7 days after the operation. EEG activity was recorded for 20 hrs using Biopac Systems amplifiers (Biopac Systems EEG100C) and AcqKnowledge 4.2 EEG Acquisition and analyzed with the Reader Software (Biopac Systems).

A total of 150 hrs of data was analyzed, 54 hrs for nTg mice and 96 hrs for APP/PS1 transgenic mice. Six hrs of recording were analyzed for each animal. All signals were filtered with a FIR notch filter at 60 Hz and its corresponding harmonics and with a bandpass filter from 0.1 to 150 Hz. Spikes were defined as a sharp amplitude deflection lasting 20-70 ms. EEG analysis was performed using MATLAB (Mathworks 2020a). Signals were clipped into 10-minute segments and determination of epileptiform activity and quantification of interictal spike activity were performed using a semi-automated analysis with p-operator algorithms described in (see, for example, Majumdar K K, and Vardhan P. Automatic seizure detection in ECOG by differential operator and windowed variance. IEEE Trans Neural Syst Rehabil Eng. 2011, 19 (4): 356-65; and Pizarro D, Ilyas A, Toth E, Romeo A, Riley K O, Esteller R, et al. Automated detection of mesial temporal and temporoperisylvian seizures in the anterior thalamic nucleus. Epilepsy research. 2018, 146:17-20). P-operator algorithm was used with a sliding window of 1 second. When p-operator algorithm detected a spike, all signals were visually inspected to include in the analysis only those that were true positives in order to avoid non-epileptic artifacts caused by electrical noise (e.g., grooming) during spike selection. Spectral content of EEG signals were calculated using short-time Fourier transform (FFT).

### Statistical Analysis

Seizure scores were analyzed by two-way ANOVA with multiple comparisons test. Statistical analyses were performed using Prism GraphPad software. For EEG data, a repeated measure ANOVA was used to calculate the statistical difference of the spike count per 10 minutes, among different treatments between nTg and APP/PS1 mice. Statistical tests were performed on IBM SPSS. Other data were analyzed with Student's t-tests, one-way or two-way ANOVA with multiple comparisons test using GraphPad software. p<0.05 was considered statistically significant.

### The A1R-Mediated Anticonvulsant Effect is Regulated by the Neurabin/RGS4 Complex and is Particularly Sensitive to a Change in Neurabin Levels

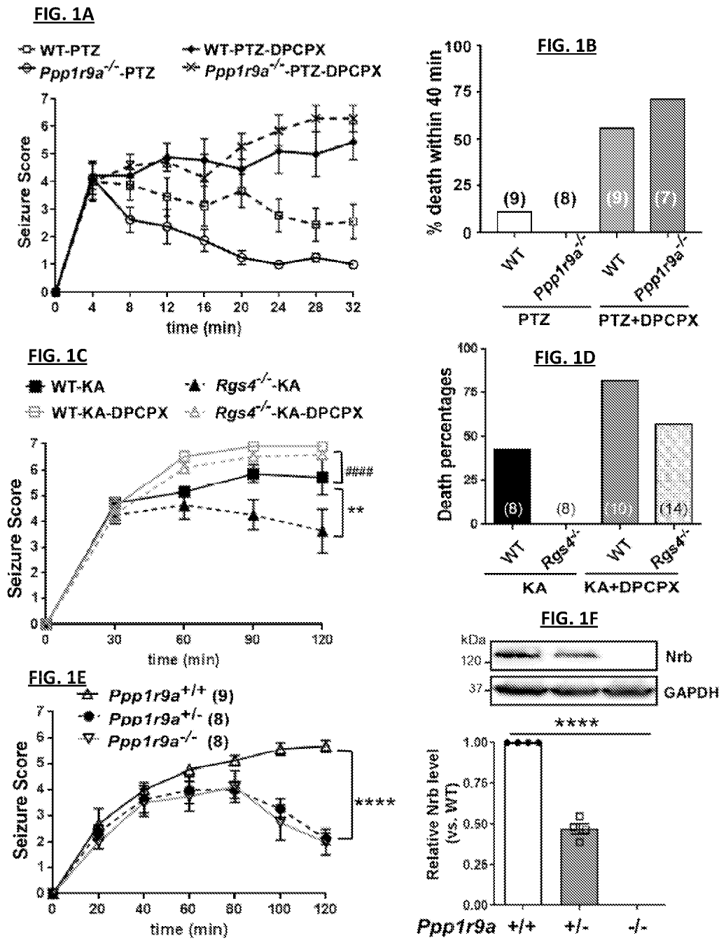

An A1R/neurabin/RGS4 complex has been identified that downregulates A1R signaling in response to endogenous adenosine. Consistently, the A1R-mediated anticonvulsant effect against kainate insult has been shown to be enhanced in neurabin deficient (Ppp1r9a^{-/-}) mice. Herein, this mechanism was tested in another chemoconvulsant model, pentylenetetrazol (PTZ)-induced seizure. PTZ is a GABAA receptor antagonist that induces a rapid seizure response, which peaked at 4 minutes and lasted over 30 minutes in WT mice, as shown in FIG. 1A. About 10% of PTZ-treated WT mice died within 40 minutes, as shown in FIG. 1B. In Ppp1r9a^{-/-} (neurabin null) mice, PTZ-induced seizures quickly declined over time after the initial peak shown in FIG. 1A, and all mice survived, as shown in FIG. 1B. When mice were co-treated with an A1R-selective blocker, DPCPX, PTZ-induced seizures in both WT and neurabin null mice continued to progress to a much more severe level than that induced by PTZ alone and there was no significant difference between the two genotypes in FIG. 1A, suggesting the requirement of A1R activation in reducing the seizure severity in neurabin null mice. The combined treatment with PTZ and DPCPX led to more than 55% death in both genotypes, shown in FIG. 1B. These data demonstrate the critical role of neurabin in attenuating the A1R-mediated anticonvulsant effect. In mice without neurabin expression, the A1R-dependent protective effect is significantly enhanced.

In Rgs4^{-/-} mice, kainate-induced seizures were significantly attenuated in both extent and duration as compared to WT mice, shown in FIG. 1C. While kainate administration at 20 mg/kg resulted in over 40% mortality in WT mice, all Rgs4^{-/-} mice survived the same insult, as shown in FIG. 1D. Furthermore, blockade of A1R by DPCPX increased the seizure severity and mortality rate in Rgs4^{-/-} mice to a level similar to that in WT mice, shown in FIG. 1C and FIG. 1D, suggesting the requirement of A1R activity in protecting Rgs4^{-/-} mice against seizures.

Kainate-induced seizure in neurabin heterozygous (Ppp1r9a^{+/-}) mice was examined. The severity and progression of kainate-induced seizures in Ppp1r9a^{+/-} mice were reduced to a level similar to that in Ppp1r9a^{-/-} mice, as shown in FIG. 1E. It was confirmed that, in heterozygous mice, the neurabin expression level was about 50% of the WT level, shown in FIG. 1F. On the other hand, 50% reduction in RGS4 levels has no effect on the A1R-dependent anticonvulsant response (average seizure severity scores are 5.7 and 5.6 in WT and Rgs4^{+/-} mice, respectively, N=5-8/group). These data suggest that the A1R-mediated anticonvulsant effect is particularly sensitive to neurabin-mediated regulation; a 50% reduction in neurabin levels can sufficiently lead to enhancement of the A1R-mediated anti-seizure effect. These data also strongly support that disruption of the A1R-neurabin interaction would be an effective means for seizure control.

### Neurabin and Rgs4 Attenuate A1R-Mediated Inhibition of Synaptic Transmission

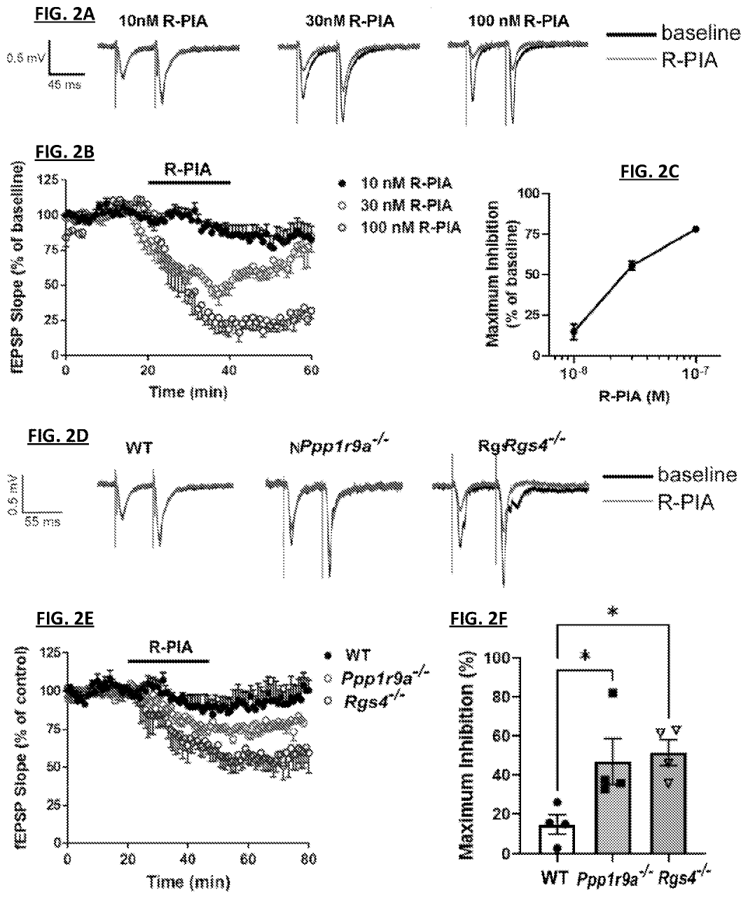

Adenosine A1R elicits anticonvulsant effects through inhibition of synaptic transmission. As shown in FIG. 2A, FIG. 2B, and FIG. 2C, an A1R agonist, R-PIA, dose-dependently inhibited field excitatory postsynaptic potential (fEPSP) at the hippocampal CA3-CA1 synapses in WT mice. 100 nM R-PIA resulted in about 75% inhibition of baseline transmission as shown in FIG. 2B and FIG. 2C. The impact of loss of neurabin or RGS4 expression on A1R-mediated depression of synaptic neurotransmission in hippocampal slices was then examined. In hippocampal slices prepared from Ppp1r9a^{-/-} or Rgs4^{-/-} mice, the same concentration of R-PIA induced a significantly higher level of inhibition of fEPSP at the CA3-CA1 synapse than in hippocampal slices from WT mice, shown in FIG. 2D, FIG. 2E, and FIG. 2F. These data demonstrate that A1R-mediated synaptic inhibition is enhanced in the absence of neurabin and RGS4 expression. Such enhancement would underlie the greater anticonvulsant effects by A1R in mice lacking these proteins.

### Neurabin Reduces Both the Response Sensitivity and Duration of A1R-Mediated Neuroprotective Akt Signaling in Neurons

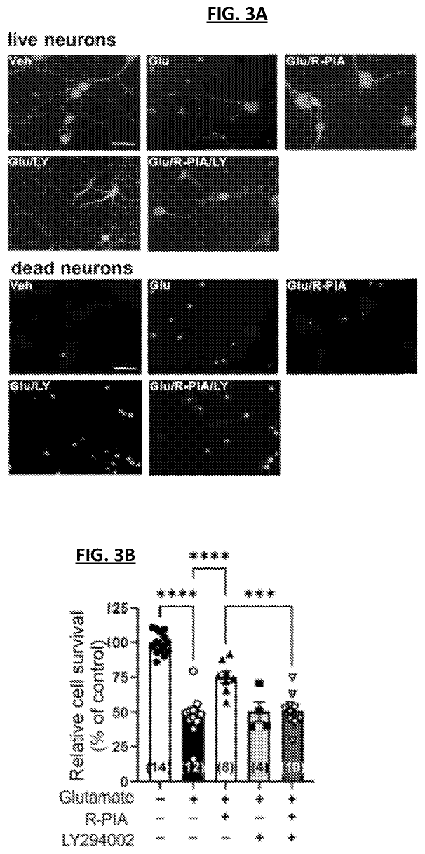

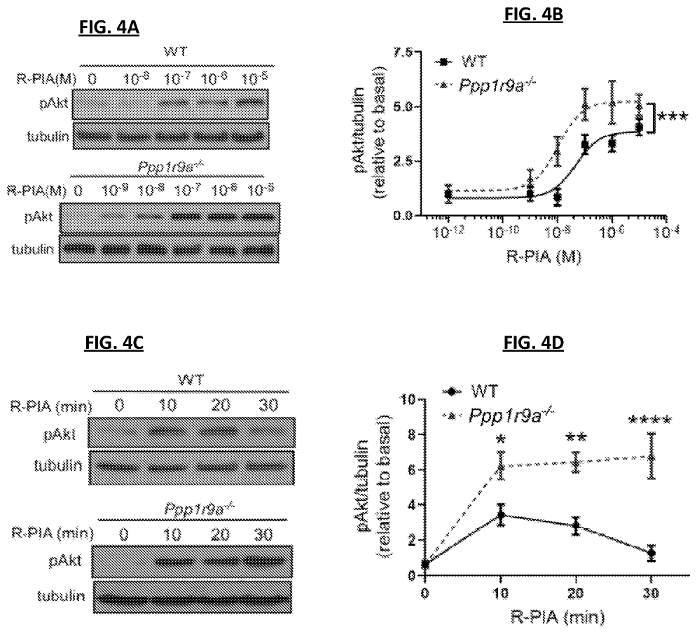

The serine-threonine protein kinase Akt is an important neurotrophic signaling component that exerts protection against neuronal death. In primary cultured hippocampal neurons, an inhibitor of phosphoinositide 3-kinase (PI3K)/Akt signaling, LY294002, abolished the protective effect of the A1R agonist R-PIA on glutamate-induced neuronal death, shown in FIG. 3A and FIG. 3B, indicating the requirement of the PI3K/Akt pathway in A1R-elicited neuroprotection. A1R-mediated Akt activation was further examined in primary neurons derived from WT and neurabin deficient (Ppp1r9a^{-/-}) mice. In WT neurons, stimulation of A1R led to a dose-dependent increase of Akt phosphorylation at Thr 308 in the activation loop. In neurons lacking neurabin expression, the same dose of R-PIA induced a significantly higher level of Akt phosphorylation compared to that in WT neurons, shown in FIG. 4A and FIG. 4B, suggesting that the response sensitivity of A1R in inducing Akt signaling is enhanced in the absence of neurabin expression. Furthermore, A1R-mediated Akt activation was markedly prolonged in neurons without neurabin expression as compared to WT neurons. While Akt activation was desensitized after 30 min stimulation with R-PIA in WT neurons, no apparent reduction in phospho-Akt (i.e. active Akt) levels was observed at the 30-mintime point in neurons lacking neurabin, as shown in FIG. 4C and FIG. 4D. Taken together, these data demonstrate that neurabin reduces A1R-dependent neuroprotective Akt signaling in neurons.

### The A1R C-Terminal Peptide Blocks the A1R-Neurabin Interaction and Abolishes Neurabin-Mediated Inhibition of A1R Signaling

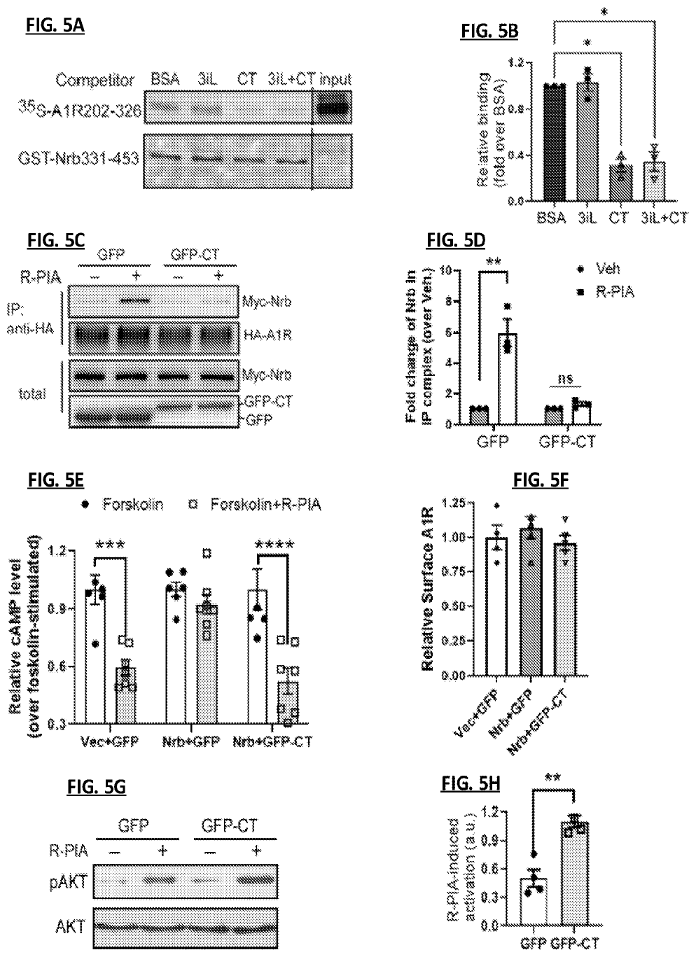

The in vitro and in vivo data described above collectively suggest that targeting the A1R-neurabin interaction represents an attractive strategy to specifically enhance A1R-mediated anti-seizure and neuroprotective effects. Therefore blockers that could disrupt the direct interaction between A1R and neurabin were investigated. The C-tail and 3i loop of A1R are both involved in binding with neurabin aa331-453 (Nrb331-453). Peptides that carry the sequences of the A1R C-tail (aa293-326) and 3i loop (aa202-235), respectively, were synthesized and tested their ability to disrupt the A1R-neurabin interaction. The addition of the C-tail peptide of A1R (A1R-CT) markedly reduced the direct interaction between the A1R (aa202-326) with Nrb331-453 in in vitro pull-down assays, shown in FIG. 5A and FIG. 5B. However, the presence of 3i loop peptide had no effect on the A1R-neurabin interaction. These data suggest that the A1R-CT peptide can act as an effective blocker to disrupt the direct interaction between A1R and neurabin. This notion is further supported by data collected using intact cells, where expression of GFP-fused A1R C-tail (GFP-CT) abolished the interaction between the full-length A1R and neurabin, as shown in FIG. 5C and FIG. 5D.

Next, the effect of the A1R-CT peptide on A1R signaling was examined. In CHO cells, in which endogenous neurabin expression could not be detected, expression of exogenous neurabin diminished A1R-induced inhibition of cAMP, shown in FIG. 5E. When GFP-tagged A1R-CT (GFP-CT) was co-expressed with neurabin, A1R-mediated CAMP inhibition was fully restored. Expression of GFP-CT did not alter surface expression of A1R (FIG. 5F). The effect of GFP-A1R-CT on A1R-mediated Akt activation was also tested. In cells overexpressing GFP-A1R-CT, the level of Akt phosphorylation in response to A1R agonist R-PIA was significantly enhanced, shown in FIG. 5G and FIG. 5H. Taken together, these data suggest that the neurabin-mediated attenuation of A1R signaling can be abolished by the expression of the A1R-CT peptide. Therefore, a peptide blocker (i.e., the A1R-CT peptide) that effectively disrupts the A1R-neurabin interaction and enhances A1R signaling was identified.

### The A1R C-Tail Peptide Displays Prominent Protective Effects Against Chemically Induced Seizures

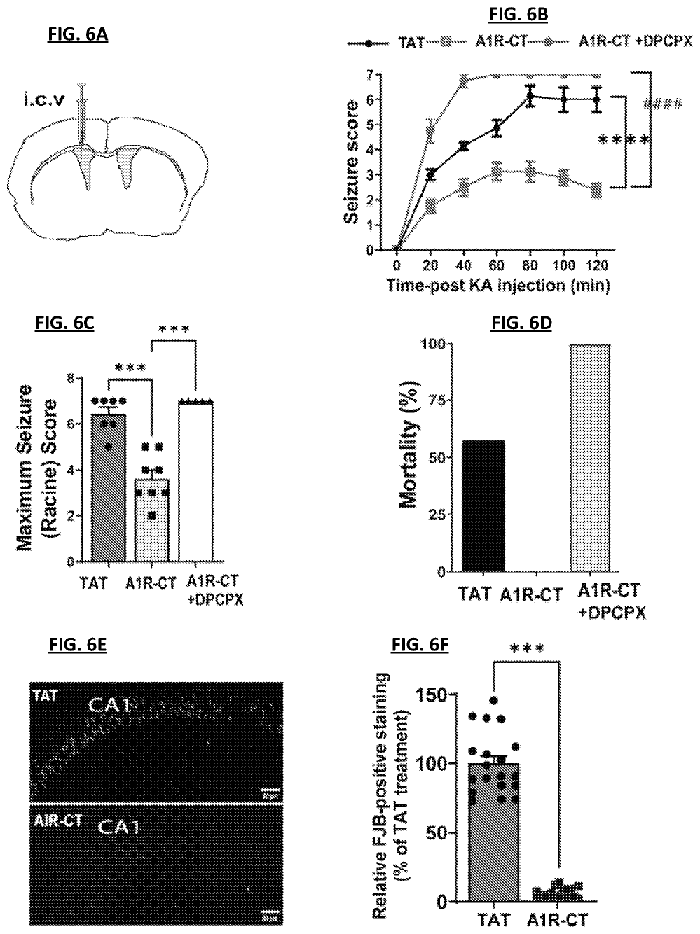

A goal in this disclosure was to improve A1R-elicited anti-seizure function in the brain. Thus, the ability of A1R-CT peptide to enhance A1R-mediated anti-seizure effects in a kainate-induced model was determined. The A1R-CT peptide was synthesized with addition of the TAT peptide sequence to facilitate diffusion into cells. The TAT-A1R-CT or TAT control peptide (500 μmol) was infused into the brain through an intracerebroventricular (icv) cannula 30 minutes prior to kainate injection, as depicted in FIG. 6A. In mice receiving the TAT-A1R-CT peptide, the severity and duration of kainate-induced seizures were significantly reduced compared to those in mice receiving TAT peptide (FIG. 6B and FIG. 6C). While 57% of mice receiving the TAT peptide died within 2 hours after kainate injection, all mice receiving the TAT-A1R-CT peptide survived, shown in FIG. 6D. When mice were co-treated with DPCPX, the anti-seizure effect of A1R-CT was no longer observed (FIG. 6B, FIG. 6C, and FIG. 6D), suggesting the requirement of A1R in mediating the protection by this peptide.

Neuronal death in the hippocampus of mice that survived kainite insult was then examined. Severe cell death was observed in the hippocampus of mice receiving the TAT peptide 7 days after kainate-induced seizures (FIG. 6E and FIG. 6F). However, in hippocampal slices prepared from TAT-A1R-CT peptide-treated mice exposed to the same level of kainate injections, little cell death was detected. These morphological findings of neuronal cell survival paralleled with seizure and survival findings shown above in FIG. 6B, FIG. 6C, and FIG. 6D, demonstrating a prominent neuroprotective effect of A1R C-tail peptide against excitotoxic cell death.

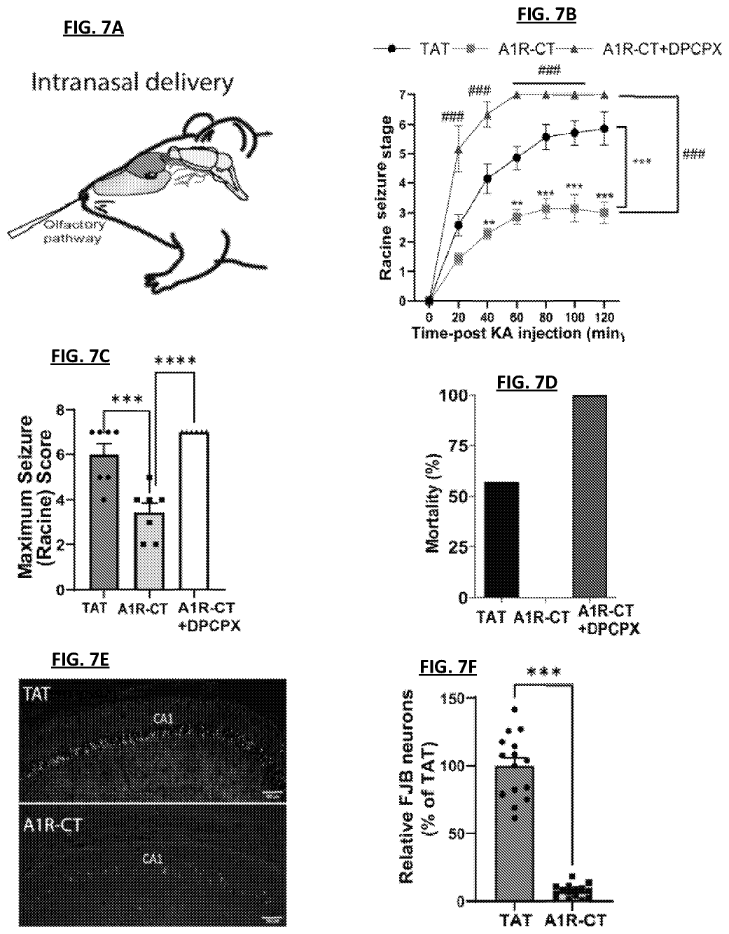

Intranasal delivery allows peptides to rapidly reach the brain in a non-invasive way. The ability of intranasal administration of the TAT-A1R-CT peptide to also elicit anti-seizure effects was determined. The TAT-A1R-CT or TAT peptide (42 nmol) was delivered into the intranasal cavity 45 min prior to kainate injection (FIG. 7A). In mice receiving the TAT-A1R-CT peptide, the severity and duration of kainate-induced seizures were significantly reduced when compared to those in mice receiving TAT peptide (FIG. 7B and FIG. 7C). All mice receiving TAT-A1R-CT peptide survived in contrast to the 60% death rate caused by kainate in mice receiving the TAT peptide (FIG. 7D). The observed anti-seizure effect of TAT-A1R-CT peptide was abolished by co-treating mice with DPCPX (FIG. 7B, FIG. 7C, and FIG. 7D), suggesting that A1R is required to mediate the protection by this peptide. Furthermore, while kainate insult resulted in severe cell death in the hippocampus of mice receiving the TAT peptide, only limited neuronal death was detected in mice receiving the TAT-A1R-CT peptide (FIG. 7E and FIG. 7F). These data demonstrate the effectiveness of intranasal delivery of the A1R-CT peptide in protection against chemoconvulsant seizures and excitotoxic cell death.

### The A1R-Ct Peptide Reduces Spontaneous Epileptic Activity in an Ad Mouse Model

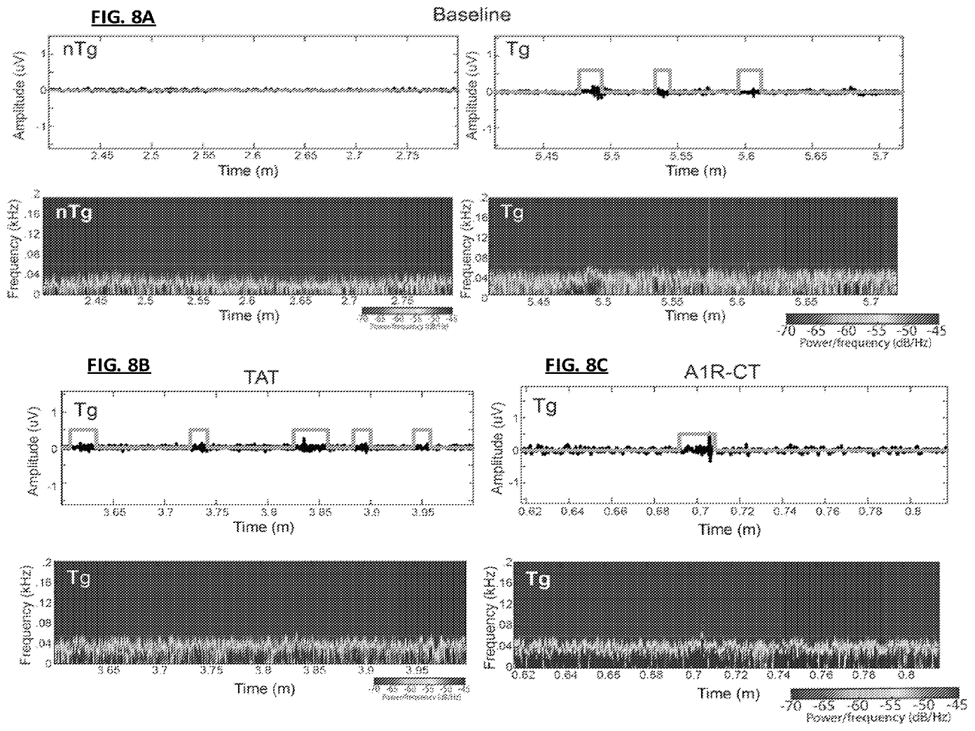

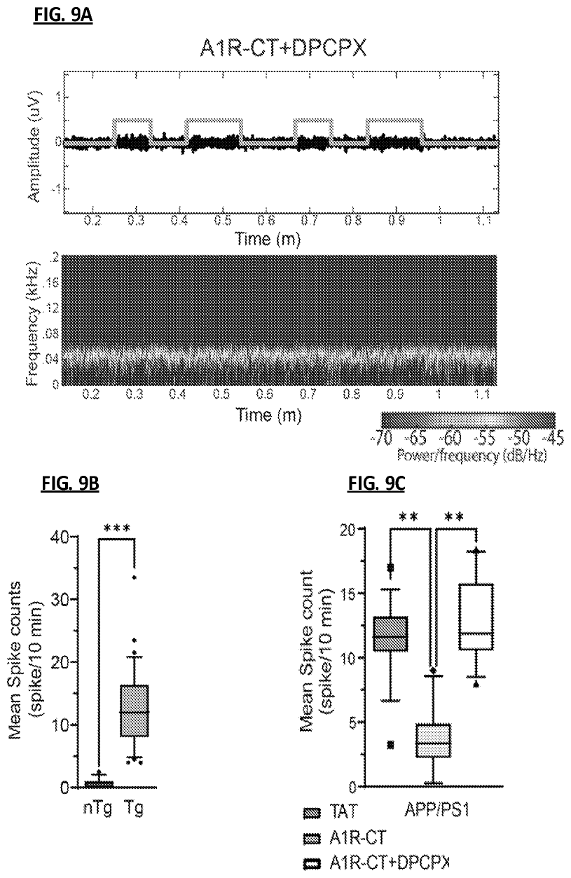

The above data demonstrate that the A1R C-tail peptide provides protection against chemoconvulsant seizures. Next, its effectiveness in reducing spontaneous epileptic activities in disease models was examined. An AD mouse model was utilized, as most current antiepileptic drugs have adverse effects on cognition or mood, making treatment of seizures in AD patients particularly challenging. APP/PS1 transgenic mice were tested using EEG recording. These mice start to develop spontaneous seizures at the onset of amyloid pathogenesis. EEG activities were monitored in freely moving 10-11 month-old APP/PS1 and their non-transgenic (nTg) littermates. APP/PS1 mice develop spontaneous epileptic activities as manifested by the appearance of epileptic spikes, and such electrographic paroxysms were not observed in their nTg littermates (FIG. 8A and FIG. 9B).

APP/PS1 mice were then treated with the TAT or TAT-A1R-CT peptide through intranasal delivery, with or without i.p. injection of DPCPX, and their brain activities were continuously monitored for 18 hrs. EEG recording of APP/PS1 mice treated with the TAT peptide displayed similar electrographic paroxysms as observed in baseline recording of these mice (FIG. 8B). However, treatment with the TAT-A1R-CT peptide led to a significant reduction in the number of epileptic spike counts compared to TAT peptide treatment in APP/PS1 mice (FIG. 8C and FIG. 9C). Intranasal delivery of the TAT-A1R-CT peptide failed to reduce epileptic activities in APP/PS1 mice that also received DPCPX (FIG. 9A and FIG. 9C), suggesting the requirement of A1R in mediating the antiepileptic effect of the TAT-A1R-CT peptide. These data suggest that the A1R-CT peptide can effectively inhibit spontaneous seizure activities associated with AD.

### Discussion

The present study identifies a novel blocking peptide, the A1R-CT peptide, which exhibits strong protective effects against both chemoconvulsant seizures and spontaneous epileptic activities in an AD mouse model. This blocking peptide disrupts the direct interaction between A1R and neurabin, a neural tissue-specific protein that negatively regulates A1R signaling and function. As a result, the A1R-CT peptide enhances A1R-mediated signaling responses and boosts endogenous adenosine-induced anti-seizure effects through A1R. Of particular significance, in all experiments performed in the studies, no exogenous A1R ligands were administered. The anticonvulsant and neuroprotective effects of this peptide blocker are achieved through the A1R in response to the endogenous adenosine released on-site, thus providing precise protection at the zone of hyperexcitability. Furthermore, neurabin is a neural-specific scaffolding protein. Therefore, this agent elicits its beneficial effects against seizures without inducing confounding outcomes due to ectopic activation of receptors in peripheral tissues and organs.

Pathological conditions such as hypoxia, ischemia and excitotoxin exposure result in hyperexcitability and cell death of neurons in the brain. The severity of neural damage is a key factor determining mortality and morbidity under these conditions. It is shown herein that A1R-mediated synaptic inhibition (FIG. 2A through FIG. 2F) is significantly enhanced in the absence of neurabin or RGS4 expression. As a result, neurabin or RGS4 deficient mice show resistance to chemoconvulsant seizures in an A1R-dependent manner (FIG. 1A through FIG. 1F). A particularly striking phenomenon is that a 50% drop of neurabin expression leads to a significant reduction in seizure severity (FIG. 1A through FIG. 1F), suggesting that endogenous adenosine-elicited anticonvulsant effects through A1R would be sensitive to modulation of the A1R-neurabin interaction. In addition, it was determined that A1R-mediated neuroprotective Akt signaling is both enhanced and prolonged in the absence of neurabin (FIG. 3A through 4D). These findings motivated the search for blockers that could disrupt the A1R-neurabin interaction, which would serve as an effective means to enhance A1R-mediated anti-seizure and neuroprotective effects.

In the disclosure herein it is demonstrated that the peptide consisting of the A1R C-tail sequence, but not the 3i loop sequence, effectively blocks the direct interaction between A1R and neurabin and reverses neurabin-mediated inhibition of A1R signaling (FIG. 5A through FIG. 5H). Significantly, when directly delivered into the brain, this A1R-CT peptide shows strong anti-seizure and neuroprotective effects in an A1R-dependent fashion (FIG. 6A through FIG. 6F), providing direct evidence for the effectiveness of blocking neurabin-A1R interaction as a means for seizure control.

To demonstrate the strong potential of this peptide as a therapeutic agent for seizure control, it was shown that non-invasive intranasal delivery of this peptide displays robust anti-seizure and neuroprotective effects against kainate (FIG. 7A through FIG. 7F), and reduces epileptic spikes in an AD mouse model. Currently available anticonvulsant drugs act through activation of GABAergic transmission or inhibition of Na⁺ or Ca²⁺ channels. If successfully developed for human use, the A1R-CT peptide would represent the first to offer a different and more targeted therapy facilitating seizure termination. Intranasal delivery of this peptide may also provide a new option for seizure rescue treatments. Currently, there are only three seizure rescue medications, diazepam rectal gel (Diastat®), diazepam nasal spray (Valtoco®) and midazolam nasal spray (Nayzilam®).

In addition to being caused by acute insults to the brain, epileptic seizures are a common comorbidity of chronic neurodegenerative diseases, including AD, the most common type of dementia. Herein, the effectiveness of the A1R-CT peptide in suppressing seizures in an AD animal model, APP/PS1 mice, was examined. The data demonstrate that intranasal delivery of the A1R-CT peptide effectively reduces the spontaneous spike frequency in the APP/PS1 model (FIG. 8A through 9C). The studies disclosed herein thus provides strong preclinical evidence supporting that noninvasive delivery of the A1R-CT peptide is effective in treating AD-related seizures.

In summary, a novel peptide blocker of the A1R-neurabin interaction, the A1R-CT peptide, was developed. The A1R-CT peptide displays strong protective effects against both chemoconvulsant and AD-related spontaneous seizures. The anticonvulsant and neuroprotective effects of this peptide are in response to the endogenous adenosine released on-site and on-demand, thus avoiding ectopic activation of receptors in other tissues and organs. The A1R-CT peptide thus represents a promising therapeutic intervention for seizure control under various pathological conditions. The demonstrated effectiveness of intranasal delivery of this agent makes it particularly attractive and clinically relevant as a potential novel seizure rescue treatment. Furthermore, this peptide represents the first agent that specifically enhances A1R functions in the CNS, and has potential use for the treatment of other neurological disorders in which A1R function is relevant.

### G. Bibliography

1. Devinsky O, Vezzani A, O'Brien T J, Jette N, Scheffer I E, de Curtis M, et al. Epilepsy. Nat Rev Dis Primers. 2018, 4:18024
2. Kotloski R J, Dowding J, Hermann B P, and Sutula T P. Epilepsy and aging. Handb Clin Neurol. 2019, 167:455-75
3. Asadollahi M, Atazadeh M, and Noroozian M. Seizure in Alzheimer's Disease: An Underestimated Phenomenon. Am J Alzheimers Dis Other Demen. 2019, 34 (2): 81-8
4. Vossel K A, Tartaglia M C, Nygaard H B, Zeman A Z, and Miller B L. Epileptic activity in Alzheimer's disease: causes and clinical relevance. Lancet Neurol. 2017, 16 (4): 311-22
5. Dingledine R, Varvel N H, and Dudek F E. When and how do seizures kill neurons, and is cell death relevant to epileptogenesis? Advances in experimental medicine and biology. 2014, 813:109-22
6. Elger C E, Helmstaedter C, and Kurthen M. Chronic epilepsy and cognition. Lancet Neurol. 2004, 3 (11): 663-72
7. Bonnett L J, Powell G A, Tudur Smith C, and Marson A G. Breakthrough seizures-Further analysis of the Standard versus New Antiepileptic Drugs (SANAD) study. PloS one. 2017, 12 (12): e0190035
8. Liu J, Wang L N, Wu L Y, and Wang Y P. Treatment of epilepsy for people with Alzheimer's disease. Cochrane Database Syst Rev. 2016, 11: CD011922
9. Sebastiao A M, and Ribeiro J A. Adenosine receptors and the central nervous system. HandbExpPharmacol. 2009, (193): 471-534
10. Dunwiddie T V, and Masino S A. The role and regulation of adenosine in the central nervous system. AnnuRevNeurosci. 2001, 24:31-55
11. Stone T W. Purines and neuroprotection. AdvExpMedBiol. 2002, 513:249-80; Boison D. Adenosine as a neuromodulator in neurological diseases. CurrOpinPharmacol. 2008, 8 (1): 2-7
12. Jacobson K A, and Gao Z G. Adenosine receptors as therapeutic targets. NatRevDrug Discov. 2006, 5 (3): 247-64
13. Chung H J, Ge W P, Qian X, Wiser O, Jan Y N, and Jan L Y. G protein-activated inwardly rectifying potassium channels mediate depotentiation of long-term potentiation ProcNatlAcadSciUSA. 2009, 106 (2): 635-40
14. Wetherington J P, and Lambert N A. Differential desensitization of responses mediated by presynaptic and postsynaptic A1 adenosine receptors. J Neurosci. 2002, 22 (4): 1248-55
15. Wu L G, and Saggau P. Adenosine inhibits evoked synaptic transmission primarily by reducing presynaptic calcium influx in area CA1 of hippocampus. Neuron. 1994, 12 (5): 1139-48
16. Badimon A, Strasburger H J, Ayata P, Chen X, Nair A, Ikegami A, et al. Negative feedback control of neuronal activity by microglia. Nature. 2020, 586 (7829): 417-23
17. Boison D. Adenosine-based cell therapy approaches for pharmacoresistant epilepsies. NeurodegenerDis. 2007, 4 (1): 28-33
18. Pedata F, Dettori I, Coppi E, Melani A, Fusco I, Corradetti R, et al. Purinergic signaling in brain ischemia. Neuropharmacology. 2016, 104:105-30
19. Bekar L, Libionka W, Tian G F, Xu Q, Torres A, Wang X, et al. Adenosine is crucial for deep brain stimulation-mediated attenuation of tremor. Nature medicine. 2008, 14 (1): 75-80
20. Chen Y, Liu Y, Cottingham C, McMahon L, Jiao K, Greengard P, et al. Neurabin scaffolding of adenosine receptor and RGS4 regulates anti-seizure effect of endogenous adenosine. J Neurosci. 2012, 32 (8): 2683-95
21. Chen Y, Booth C, Wang H, Wang R X, Terzi D, Zachariou V, et al. Effective Attenuation of Adenosine A1R Signaling by Neurabin Requires Oligomerization of Neurabin. Mol Pharmacol. 2017, 92 (6): 630-9
22. Stratinaki M, Varidaki A, Mitsi V, Ghose S, Magida J, Dias C, et al. Regulator of G protein signaling 4 [corrected] is a crucial modulator of antidepressant drug action in depression and neuropathic pain models. Proc Natl Acad Sci USA. 2013, 110 (20): 8254-9
23. Han M H, Renthal W, Ring R H, Rahman Z, Psifogeorgou K, Howland D, et al. Brain region specific actions of regulator of G protein signaling 4 oppose morphine reward and dependence but promote analgesia. Biological psychiatry. 2010, 67 (8): 761-9
24. Avrampou K, Pryce K D, Ramakrishnan A, Sakloth F, Gaspari S, Serafini R A, et al. RGS4 Maintains Chronic Pain Symptoms in Rodent Models. J Neurosci. 2019, 39 (42): 8291-304
25. Dudek H, Datta S R, Franke T F, Birnbaum M J, Yao R, Cooper G M, et al. Regulation of neuronal survival by the serine-threonine protein kinase Akt. Science. 1997, 275 (5300): 661-5
26. Minkeviciene R, Rheims S, Dobszay M B, Zilberter M, Hartikainen J, Fulop L, et al. Amyloid beta-induced neuronal hyperexcitability triggers progressive epilepsy. J Neurosci. 2009, 29 (11): 3453-62
27. Haas H L, and Selbach O. Functions of neuronal adenosine receptors. Naunyn Schmiedebergs ArchPharmacol. 2000, 362 (4-5): 375-81
28. Cunha R A. Adenosine as a neuromodulator and as a homeostatic regulator in the nervous system: different roles, different sources and different receptors. Neurochemint. 2001, 38 (2): 107-25
29. Hesdorffer D C, Hauser W A, Annegers J F, Kokmen E, and Rocca W A. Dementia and adult-onset unprovoked seizures. Neurology. 1996, 46 (3): 727-30
30. Cook M, Baker N, Lanes S, Bullock R, Wentworth C, and Arrighi H M. Incidence of stroke and seizure in Alzheimer's disease dementia. Age Ageing. 2015, 44 (4): 695-9
31. Vossel K A, Beagle A J, Rabinovici G D, Shu H, Lee S E, Naasan G, et al. Seizures and epileptiform activity in the early stages of Alzheimer disease. JAMA Neurol. 2013, 70 (9): 1158-66
32. Cretin B, Sellal F, Philippi N, Bousiges O, Di Bitonto L, Martin-Hunyadi C, et al. Epileptic Prodromal Alzheimer's Disease, a Retrospective Study of 13 New Cases: Expanding the Spectrum of Alzheimer's Disease to an Epileptic Variant? Journal of Alzheimer's disease: JAD. 2016, 52 (3): 1125-33
33. Vossel K A, Tartaglia M C, Nygaard H B, Zeman A Z, and Miller B L. Epileptic activity in Alzheimer's disease: causes and clinical relevance. Lancet Neurol. 2017, 16 (4): 311-22
34. Samson W N, van Duijn C M, Hop W C, and Hofman A. Clinical features and mortality in patients with early-onset Alzheimer's disease. European neurology. 1996, 36 (2): 103-6
35. Romanelli M F, Morris J C, Ashkin K, and Coben L A. Advanced Alzheimer's disease is a risk factor for late-onset seizures. Archives of neurology. 1990, 47 (8): 847-50
36. Volicer L, Smith S, and Volicer B J. Effect of seizures on progression of dementia of the Alzheimer type. Dementia. 1995, 6 (5): 258-63
37. Lott I T, Doran E, Nguyen V Q, Tournay A, Movsesyan N, and Gillen D L. Down syndrome and dementia: seizures and cognitive decline. Journal of Alzheimer's Disease: JAD. 2012, 29 (1): 177-85
38. Vossel K, Ranasinghe K G, Beagle A J, La A, Ah Pook K, Castro M, et al. Effect of Levetiracetam on Cognition in Patients With Alzheimer Disease With and Without Epileptiform Activity: A Randomized Clinical Trial. JAMA Neurol. 2021, 78 (11): 1345-54
39. Palop J J, and Mucke L. Amyloid-beta-induced neuronal dysfunction in Alzheimer's disease: from synapses toward neural networks. Nat Neurosci. 2010, 13 (7): 812-8;
40. Born H A. Seizures in Alzheimer's disease. Neuroscience. 2015, 286:251-63

### H. Conclusion

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving all such objectives. Any statements in this description criticizing the prior art are not intended to limit what is claimed to exclude any aspects of the prior art.

Additionally, the disclosure shows and describes certain embodiments of the processes, machines, manufactures, compositions of matter, and other teachings disclosed, but it is to be understood that the teachings of the present disclosure are capable of use in various other combinations, modifications, and environments and is capable of changes or modifications within the scope of the teachings as expressed herein.

Any section headings herein are provided only for consistency with the suggestions of 37 C.F.R. § 1.77 or otherwise to provide organizational queues. These headings shall not limit or characterize the invention(s) set forth herein.

The following is claimed:

1. A method of treating or preventing a neurological condition in a subject in need thereof, the method comprising: increasing the activity of the adenosine A1 receptor (A1R) specifically in the neural tissue of the subject.

2. A method of treating or preventing a neurological condition in a subject in need thereof, the method comprising: reducing binding of the adenosine A1 receptor (A1R) to neurabin.

3. A method of treating or preventing a neurological condition in a subject in need thereof, the method comprising: administering to the subject a therapeutically effective amount of an agent comprising a peptide sequence from the C-terminal region of the adenosine A1 receptor (A1R) or a functional derivative thereof to the subject.

4. A method of treating or preventing a neurological condition in a subject in need thereof, the method comprising: administering to the subject a therapeutically effective amount of a nucleic acid encoding an agent, said agent comprising a peptide sequence from the C-terminal region of the adenosine A1 receptor (A1R) or a functional derivative thereof to the subject.

5. A method of modulating the interaction of the adenosine A1 receptor (A1R) with neurabin in a cell, the method comprising: contacting the cell with an agent comprising a peptide sequence from the C-terminal region of the adenosine A1 receptor (A1R).

6. A medicament for the treatment or prevention of a neurological condition comprising a therapeutically effective amount of an agent comprising a peptide sequence from the C-terminal region of the adenosine A1 receptor (A1R) or a functional derivative thereof.

7. An agent comprising a peptide sequence from the C-terminal region of the adenosine A1 receptor (A1R) or a functional derivative thereof for use in the treatment or prevention of a neurological condition.

8. A medicament for treating or preventing a neurological condition comprising as a main ingredient an agent comprising a peptide sequence from the C-terminal region of the adenosine A1 receptor (A1R) or a functional derivative thereof.

9. Use of an agent comprising a peptide sequence from the C-terminal region of the adenosine A1 receptor (A1R) or a functional derivative thereof for the manufacture of a medicament for the treatment or prevention of a neurological condition.

10. Any one of claims 1 to 4 or 6 to 9, wherein the neurological condition is a seizure disorder.

11. Any one of claims 1 to 4 or 6 to 10, wherein the neurological condition is a seizure disorder caused by neuronal excitability.

12. Any one of claims 1 to 4 or 6 to 11, wherein the neurological condition is epilepsy, brain injury, stroke, intracerebral hemorrhage, Alzheimer's disease, Parkinson's disease, Lewy body dementia.

13. Any one of claims 1 to 4 or 6 to 12, wherein the neurological condition is cell death caused by at least one of: hypoxia, ischemia, excitotoxin exposure, a traumatic injury, a chemical agent, and aglycemia.

14. Any one of claims 1-4 or 6 to 13, wherein the route of agent administration is at least one of: intranasal, intracranial, intracerebroventricular, subcutaneous, intravenous, topical, epicutaneous, oral, intraosseous, intramuscular, and pulmonary.

15. Any one of claims 1-4 or 6 to 14, wherein the route of agent administration is intranasal.

16. Any one of claims 1-4 or 6 to 14, wherein the route of agent administration is intracerebroventricular.

17. Any one of claims 1-4 or 6 to 16, wherein the agent is administered to the subject at a dosage of 1-100 mg/kg, 1.1-90 mg/kg, 1.25-80 mg/kg, 1.43-70 mg/kg, 1.67-60 mg/kg, 2-50 mg/kg, 2.5-40 mg/kg, 3.33-30 mg/kg, 5-20 mg/kg, 10 mg/kg or about any of the foregoing.

18. Any one of claims 3-17, wherein the agent comprises a cell-permeable transporter tag.

19. Any one of claims 3-18, wherein the agent comprises a cell-permeable transporter tag and a linker peptide sequence between the cell-permeable transporter tag and the C-terminal region of the A1R.

20. Any one of claims 3-19, wherein the agent comprises a cell-permeable transporter tag and a linker peptide sequence between the cell-permeable transporter tag and the C-terminal region of the A1R, and wherein the linker peptide sequence comprises 4-10 amino acid residues.

21. Any one of claims 3-20, wherein the agent comprises a cell-permeable transporter tag and a linker peptide sequence between the cell-permeable transporter tag and the C-terminal region of the A1R, and wherein the linker peptide sequence comprises the sequence GSGSGS.

22. Any one of claims 3-21, wherein the agent comprises a cell-permeable transporter tag that is a trans-activator of transcription (TAT) protein of HIV-1 or a functional derivative thereof.

23. Any one of claims 3-22 wherein the agent comprises a cell-permeable transporter tag comprising SEQ ID NO: 9 or a functional derivative thereof.

24. Any one of claims 3-23, wherein the peptide sequence from the C-terminal region of the A1R contains up to 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acid residues.

25. Any one of claims 3-24 wherein the peptide sequence from the C-terminal region of the A1R contains at least 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acid residues.

26. Any one of claims 3-25, wherein the peptide sequence from the C-terminal region of the A1R contains about 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, or 10 amino acid residues.

27. Any one of claims 3-26, wherein the peptide sequence from the C-terminal region of the A1R contains about 36 amino acid residues.

28. Any one of claims 3-27, wherein the peptide sequence from the C-terminal region of the A1R comprises one of SEQ ID NOS: 1-7, and 12 or a functional derivative of any one of the foregoing.

29. Any one of claims 3-28, wherein the peptide sequence from the C-terminal region of the A1R comprises one of SEQ ID NOS: 1-7, and 12 or a sequence having at least 70, 75, 80, 85, 90, 91, 92, 93, 94, 95, 96, 97, 98, or 99% sequence identity with one of SEQ ID NOS: 1-7, 11, and 12.

30. Any one of claims 3-29, wherein the peptide sequence from the C-terminal region of the A1R comprises SEQ ID NO: 1.

31. Any one of claims 3-30, wherein the peptide sequence from the C-terminal region of the A1R comprises SEQ ID NO: 4.

32. Any one of claims 3-31, wherein the peptide sequence from the C-terminal region of the A1R comprises SEQ ID NO: 12.

33. Any one of claims 3 to 32, wherein: the agent comprises a cell-permeable transporter tag and a linker peptide sequence between the cell-permeable transporter tag and the C-terminal region of the A1R; wherein the peptide sequence from the C-terminal region of the A1R comprises SEQ ID NO: 12; wherein the cell-permeable transporter tag is the trans-activator of transcription (TAT) protein of HIV-1 or a functional derivative thereof; and wherein the linker peptide sequence comprises 3-9, 4-8, or 5-7 amino acid residues.

34. Any one of claims 3-33, wherein the agent comprises a polypeptide sequence of SEQ ID NO: 10 or a functional derivative thereof.

35. Any one of claims 3-34, wherein the agent comprises a polypeptide sequence of SEQ ID NO: 11 or a functional derivative thereof.

36. Any one of claims 3-34, wherein the agent comprises a polypeptide sequence having at least 70, 80, 85, 90, 91, 92, 93, 94, 95, 96, 97, 98, or 99% sequence identity with at least one of SEQ ID NOS: 10 and 11.

37. Any one of claims 3-36, wherein the agent is formulated for at least one of intranasal, intracranial, intracerebroventricular, subcutaneous, intravenous, topical, epicutaneous, oral, intraosseous, intramuscular, and pulmonary administration.

38. Any one of claims 3-37, wherein the agent is formulated for intranasal administration.

39. A nucleic acid encoding any one of the agents of claims 3-38.

40. A nucleic acid that is complementary to the nucleic acid of claim 39.

41. A vector comprising the nucleic acid of any one of claims 38-39.

42. A genetically modified cell comprising the nucleic acid of any one of claims 38-39.
